SEQUENCE LISTING (51 sequences)

SEQ ID NO: 1 - protein, 43 aa, Artificial Sequence
  Synthesized Peptide
  SITE (3)..(3): Xaa stands for Ala,Arg,Gln,Val,Leu,Lys,Phe,or His.
  SITE (4)..(4): Xaa stands for Glu or His.
  SITE (6)..(6): Xaa stands for Ala,Gln,Val,Tyr,Arg,Leu,or Glu.
  SITE (7)..(7): Xaa stands for Ala or Ser.
  SITE (9)..(9): Xaa stands for Glu,Leu,His,Arg,Gly,or Lys.
  SITE (10)..(10): Xaa stands for Ala,Lys,Met,Arg,Gly,or Tyr.
  SITE (11)..(11): Xaa stands for Glu or Asp.
  SITE (13)..(13): Xaa stands for Ala,Gln,Arg,Tyr,Glu,Lys,or Gly.
  SITE (14)..(14): Xaa stands for Ala or Ile.
  SITE (16)..(16): Xaa stands for Glu,Gly,Tyr,Gln,Arg,Lys,or Met.
  SITE (18)..(18): Xaa stands for Phe,Ala,Gln,Val,Gly,Ile,Asn,Arg,Met,Asp,Leu,Ser,Pro,Lys,or Glu.
  SITE (19)..(19): Xaa stands for Gly,Val,Lys,Thr,Pro,Arg,Met,Asn,Glu,Asp,Ser,Cys,Trp,His,or Ala.
  SITE (20)..(20): Xaa stands for Gly,Arg,Glu,Phe,Ala,Lys,Pro,Asn,Leu,Val,Met,Asp,His,Thr,or Tyr.
  SITE (21)..(21): Xaa stands for Val,Asp,Leu,Met,Ser,Thr,Ala,Asn,His,Gly,Phe,Glu,Arg,Pro,Tyr,or Lys.
  SITE (22)..(22): Xaa stands for Val,Lys,Tyr,Thr,Asn,Gly,Asp,Glu, Pro,Phe,Gln,His,or Ile.
  SITE (23)..(23): Xaa stands for Tyr,Arg,His,Leu,Pro,Asn,Glu,Met, Ala,Gly,Val,Ile,or Ser.
  SITE (24)..(24): Xaa stands for Ser,Lys,Met,Gly,His,Gln,Thr,Val,Cys,Leu,Asn,Ala,Glu,Pro,or Arg.
  SITE (25)..(25): Xaa stands for Cys,Ser,Ala,Thr,Arg,Glu,Asn,Gln,Gly,Lys,Tyr,Pro,Leu,Val,or Met.
  SITE (26)..(26): Xaa stands for Glu,Lys,Ser,Val,Leu,Arg,Gly,Ile,Phe,Thr,Ala,His,Gln,Asn,Met,or Tyr.
  SITE (27)..(27): Xaa stands for Trp or Gly.
  SITE (28)..(28): Xaa stands for Gln,Lys,Phe,or His.
  SITE (29)..(29): Xaa stands for Ala or Leu.
  SITE (30)..(30): Xaa stands for Val,Met,Asp,Tyr,Lys,Leu,Ile,Arg,Ser,Gln,Gly,Glu,His,Phe,Asn,or Ala.
  SITE (31)..(31): Xaa stands for Tyr,Met,Arg,Ser,Lys,His,Gly,Leu, Asn,Glu,Val,Asp,or Ala.
  SITE (33)..(33): Xaa stands for His,Phe,Tyr,Lys,Ile,Gln,Met,or Val.
  SITE (34)..(34): Xaa stands for Tyr,Trp,Gly,Leu,Asn,Asp,Gln,Glu,Met,Phe,Arg,His,Ser,Lys,Val,or Ile.
  SITE (35)..(35): Xaa stands for Lys,Arg,or Gln.
  SITE (37)..(37): Xaa stands for Met,Glu,Trp,Val,His,Ser,Asn,Ile,Gln,Tyr,Gly,Leu,Arg,Phe,or Ala.
  SITE (38)..(38): Xaa stands for Arg,Leu,Val,Ser,His,Tyr,Phe,Asn,Lys,Gln,Trp,Glu,Ile,Gly,or Ala.
  SITE (40)..(40): Xaa stands for Phe,Arg,Asp,Gln,Met,Lys,Leu,or His.
  SITE (41)..(41): Xaa stands for Gln,Gly,Tyr,Arg,Asn,Ile,Ser,His,Glu,Met,Leu,Asp,Cys,Trp,Val,or Phe.
CAXXLXXLXXXLXXLXGXXXXXXXXXXXXXXLXXXLXXLXXAC

SEQ ID NO: 2 - protein, 43 aa, Artificial Sequence
  Synthesized Peptide
  SITE (18)..(18): Xaa stands for Phe,Ala,Gln,Val,Gly,Ile,Asn,Arg,Met,Asp,Leu,Ser,Pro,Lys,or Glu.
  SITE (19)..(19): Xaa stands for Gly,Val,Lys,Thr,Pro,Arg,Met,Asn,Glu,Asp,Ser,Cys,Trp,His,or Ala.
  SITE (20)..(20): Xaa stands for Gly,Arg,Glu,Phe,Ala,Lys,Pro,Asn,Leu,Val,Met,Asp,His,Thr,or Tyr.
  SITE (21)..(21): Xaa stands for Val,Asp,Leu,Met,Ser,Thr,Ala,Asn,His,Gly,Phe,Glu,Arg,Pro,Tyr,or Lys.
  SITE (22)..(22): Xaa stands for Val,Lys,Tyr,Thr,Asn,Gly,Asp,Glu, Pro,Phe,Gln,His,or Ile.
  SITE (23)..(23): Xaa stands for Tyr,Arg,His,Leu,Pro,Asn,Glu,Met, Ala,Gly,Val,or Ile.
  SITE (24)..(24): Xaa stands for Ser,Lys,Met,Gly,His,Gln,Thr,Val,Cys,Leu,Asn,Ala,Glu,or Arg.
  SITE (25)..(25): Xaa stands for Cys,Ser,Ala,Thr,Arg,Glu,Asn,Gln,Gly,Lys,Tyr,Pro,Leu,Val,or Met.
  SITE (26)..(26): Xaa stands for Glu,Lys,Ser,Val,Leu,Arg,Gly,Ile,Phe,Thr,Ala,His,Gln,Asn,Met,or Tyr.
  SITE (27)..(27): Xaa stands for Trp or Gly.
  SITE (28)..(28): Xaa stands for Gln or Lys.
  SITE (29)..(29): Xaa stands for Ala or Leu.
  SITE (30)..(30): Xaa stands for Val,Met,Asp,Tyr,Lys,Leu,Ile,Arg, Ser,Gln,Gly,or Glu.
  SITE (31)..(31): Xaa stands for Tyr,Met,Arg,Ser,Lys,His,Gly,Leu, Asn,Glu,or Val.
  SITE (34)..(34): Xaa stands for Tyr,Trp,Gly,Leu,Asn,Asp,Gln,Glu,Met,Phe,Arg,His,Ser,Lys,or Val.
  SITE (35)..(35): Xaa stands for Lys or Arg.
  SITE (37)..(37): Xaa stands for Met,Glu,Trp,Val,His,Ser,Asn,Ile, Gln,Tyr,Gly,Leu,or Arg.
  SITE (38)..(38): Xaa stands for Arg,Leu,Val,Ser,His,Tyr,Phe,Asn,Lys,Gln,Trp,Glu,Ile,or Gly.
  SITE (41)..(41): Xaa stands for Gln,Gly,Tyr,Arg,Asn,Ile,Ser,His,Glu,Met,Leu,Asp,Cys,Trp,or Val.
CAAELAALEAELAALEGXXXXXXXXXXXXXXLKXXLXXLKXAC

SEQ ID NO: 3 - protein, 43 aa, Artificial Sequence
  Synthesized Peptide
  SITE (3)..(3): Xaa stands for Ala,Arg,Gln,Val,Leu,Lys,Phe,or His.
  SITE (4)..(4): Xaa stands for Glu or His.
  SITE (6)..(6): Xaa stands for Ala,Gln,Val,Tyr,Arg,Leu,or Glu.
  SITE (7)..(7): Xaa stands for Ala or Ser.
  SITE (9)..(9): Xaa stands for Glu,Leu,His,Arg,Gly,or Lys.
  SITE (10)..(10): Xaa stands for Ala,Lys,Met,Arg,Gly,or Tyr.
  SITE (11)..(11): Xaa stands for Glu or Asp.
  SITE (13)..(13): Xaa stands for Ala,Gln,Arg,Tyr,Glu,Lys,or Gly.
  SITE (14)..(14): Xaa stands for Ala or Ile.
  SITE (16)..(16): Xaa stands for Glu,Gly,Tyr,Gln,Arg,Lys,or Met.
  SITE (18)..(18): Xaa stands for Gly or Pro.
  SITE (19)..(19): Xaa stands for Gly,Pro,Asp,or Ser.
  SITE (20)..(20): Xaa stands for Gly or Arg.
  SITE (21)..(21): Xaa stands for Val,Gly,or Pro.
  SITE (22)..(22): Xaa stands for Gly or His.
  SITE (23)..(23): Xaa stands for Leu,Gly,or Ser.
  SITE (24)..(24): Xaa stands for Pro or Arg.
  SITE (25)..(25): Xaa stands for Arg or Pro.
  SITE (26)..(26): Xaa stands for Leu or Arg.
  SITE (27)..(27): Xaa stands for Gly.
  SITE (28)..(28): Xaa stands for Lys,Phe,or His.
  SITE (30)..(30): Xaa stands for Met,Tyr,Leu,Arg,Gln,Gly,His,Phe, Asn,or Ala.
  SITE (31)..(31): Xaa stands for Arg,Leu,Asn,Asp,or Ala.
  SITE (33)..(33): Xaa stands for His,Phe,Tyr,Lys,Gln,Met,or Val.
  SITE (34)..(34): Xaa stands for Tyr,Leu,Phe,Arg,Val,or Ile.
  SITE (35)..(35): Xaa stands for Lys or Gln.
  SITE (37)..(37): Xaa stands for His,Tyr,Leu,Phe,or Ala.
  SITE (38)..(38): Xaa stands for Leu,Asn,Glu,Gly,or Ala.
  SITE (40)..(40): Xaa stands for Phe,Arg,Asp,Gln,Met,Lys,Leu,or His.
  SITE (41)..(41): Xaa stands for Gly,Asn,Ile,His,Leu,Trp,Val,or Phe.
CAXXLXXLXXXLXXLXGXXXXXXXXXXXLXXLXXXLXXLXXAC

SEQ ID NO: 4 - protein, 43 aa, Artificial Sequence
  Synthesized Peptide
CAAELAALEAELAALEGFGGVVYSCEWQAVYLKYKLMRLKQAC

SEQ ID NO: 5 - protein, 43 aa, Artificial Sequence
  Synthesized Peptide
CAAELAALEAELAALEGAVGDKRKSKGKLMMLKQKLELLKGAC

SEQ ID NO: 6 - protein, 43 aa, Artificial Sequence
  Synthesized Peptide
CAAELAALEAELAALEGVGRLYHMASGKLDRLKGKLMVLKYAC

SEQ ID NO: 7 - protein, 43 aa, Artificial Sequence
  Synthesized Peptide
CAAELAALEAELAALEGVKELTLGTVGKLMSLKYKLWSLKQAC

SEQ ID NO: 8 - protein, 43 aa, Artificial Sequence
  Synthesized Peptide
CAAELAALEAELAALEGQTFMNPHRLGKLDKLKLKLVHLKRAC

SEQ ID NO: 9 - protein, 43 aa, Artificial Sequence
  Synthesized Peptide
CAAELAALEAELAALEGVPGDNNGTRGKLYHLKNKLHRLKNAC

SEQ ID NO: 10 - protein, 43 aa, Artificial Sequence
  Synthesized Peptide
CAAELAALEAELAALEGGRFSGLQEKGKLYGLKNKLHYLKRAC

SEQ ID NO: 11 - protein, 43 aa, Artificial Sequence
  Synthesized Peptide
CAAELAALEAELAALEGGMATDHMNRGKLYRLKNKLHFLKIAC

SEQ ID NO: 12 - protein, 43 aa, Artificial Sequence
  Synthesized Peptide
CAAELAALEAELAALEGAKKAEEGQKGKLYRLKNKLHYLKSAC

SEQ ID NO: 13 - protein, 43 aa, Artificial Sequence
  Synthesized Peptide
CAAELAALEAELAALEGINAVPMSGRGKLYSLKNKLHYLKNAC

SEQ ID NO: 14 - protein, 43 aa, Artificial Sequence
  Synthesized Peptide
CAAELAALEAELAALEGNTANDPTASGKLKYLKYKLSNLKHAC

SEQ ID NO: 15 - protein, 43 aa, Artificial Sequence
  Synthesized Peptide
CAAELAALEAELAALEGREPSGAMGSGKLKRLKYKLQNLKHAC

SEQ ID NO: 16 - protein, 43 aa, Artificial Sequence
  Synthesized Peptide
CAAELAALEAELAALEGGDEVPHVGGGKLKMLKYQLNNLKHAC

SEQ ID NO: 17 - protein, 43 aa, Artificial Sequence
  Synthesized Peptide
CAAELAALEAELAALEGMSNHTLGSIGKLMLLKDKLESLKEAC

SEQ ID NO: 18 - protein, 43 aa, Artificial Sequence
  Synthesized Peptide
CAAELAALEAELAALEGDCLAGGCSFGKLLNLKQKLIRLKRAC

SEQ ID NO: 19 - protein, 43 aa, Artificial Sequence
  Synthesized Peptide
CAAELAALDAELAALEGARLGKMSKVGKLIKLKEKLQNLKMAC

SEQ ID NO: 20 - protein, 43 aa, Artificial Sequence
  Synthesized Peptide
CAAELAALEAELAALEGLGGFFNLRTGKLDELKMKLQHLKQAC

SEQ ID NO: 21 - protein, 43 aa, Artificial Sequence
  Synthesized Peptide
CAAELAALEAELAALEGAWVEQVNRRGKLDGLKMKLQRLKLAC

SEQ ID NO: 22 - protein, 43 aa, Artificial Sequence
  Synthesized Peptide
CAAELAALEAELAALEGVPALVGQEGGKLVLLKWKLWRLKNAC

SEQ ID NO: 23 - protein, 43 aa, Artificial Sequence
  Synthesized Peptide
CAAELAALEAELAALEGSVALGNKAGGKLRLLKLKLELLKDAC

SEQ ID NO: 24 - protein, 43 aa, Artificial Sequence
  Synthesized Peptide
CAAELAALEAELAALEGSSMDHVAYTGKLRMLKFRLELLKGAC

SEQ ID NO: 25 - protein, 43 aa, Artificial Sequence
  Synthesized Peptide
CAAELAALEAELAALEGADAFTLGSAGKLMMLKEKLNHLKHAC

SEQ ID NO: 26 - protein, 43 aa, Artificial Sequence
  Synthesized Peptide
CAAELAALEAELAALEGPHPRHHHPHGKLKKLKRKLYKLKQAC

SEQ ID NO: 27 - protein, 43 aa, Artificial Sequence
  Synthesized Peptide
CAAELAALEAELAALEGVRPETMSQVGKLLVLKHRLGQLKRAC

SEQ ID NO: 28 - protein, 43 aa, Artificial Sequence
  Synthesized Peptide
CAAELAALEAELAALEGGRDRTLELSGKLLVLKSKLHWLKHAC

SEQ ID NO: 29 - protein, 43 aa, Artificial Sequence
  Synthesized Peptide
CAAELAALEAELAALEGKEHPTLRTSGKLLHLKLKLHELKRAC

SEQ ID NO: 30 - protein, 43 aa, Artificial Sequence; identical to SEQ ID NO: 7
  Synthesized Peptide

SEQ ID NO: 31 - protein, 43 aa, Artificial Sequence
  Synthesized Peptide
CAAELAALEAELAALEGEGMSTLNRQGKLSVLKSKLLWLKHAC

SEQ ID NO: 32 - protein, 43 aa, Artificial Sequence
  Synthesized Peptide
CAAELAALEAELAALEGRTFTIGEVKGKLQGLIMKLHHLKDAC

SEQ ID NO: 33 - protein, 43 aa, Artificial Sequence
  Synthesized Peptide
CAAELAALEAELAALEGRPVRTVLTNGKLMVLKSKLQELKHAC

SEQ ID NO: 34 - protein, 43 aa, Artificial Sequence
  Synthesized Peptide
CAAELAALEAELAALEGQKHFTLGGLGKLIVLKKQLRILKCAC

SEQ ID NO: 35 - protein, 43 aa, Artificial Sequence
  Synthesized Peptide
CAAELAALEAELAALEGGAFLTVGRAGKLMMLKQKLVRLKCAC

SEQ ID NO: 36 - protein, 43 aa, Artificial Sequence
  Synthesized Peptide
CAAELAALEAELAALEGENVYTLGGLGKLMLLKYKLRELKGAC

SEQ ID NO: 37 - protein, 43 aa, Artificial Sequence
  Synthesized Peptide
CAAELAALEAELAALEGKDFRTIELVGKLVLLKYKLNNLKEAC

SEQ ID NO: 38 - protein, 43 aa, Artificial Sequence
  Synthesized Peptide
CAAELAALEAELAALEGGRTPVVTTMGKLMGLKLKLQQLKRAC

SEQ ID NO: 39 - protein, 43 aa, Artificial Sequence
  Synthesized Peptide
CAAELAALEAELAALEGETNMTVLMTGKLLVLKHKLEHLKWAC

SEQ ID NO: 40 - protein, 43 aa, Artificial Sequence
  Synthesized Peptide
CAAELAALEAELAALEGASTPTLESRGKLGSLIFKLRQLKVAC

SEQ ID NO: 41 - protein, 43 aa, Artificial Sequence
  Synthesized Peptide
CAAELAALEAELAALEGLHTLTLMQTGKLLVLKMKLRELKWAC

SEQ ID NO: 42 - protein, 43 aa, Artificial Sequence
  Synthesized Peptide
CAAELAALEAELAALEGGAPVTIMGVGKLMVLKEKLRNLKDAC

SEQ ID NO: 43 - protein, 43 aa, Artificial Sequence
  Synthesized Peptide
CAAELAALEAELAALEGSTARTLMQVGKLEMLKYKLLELKCAC

SEQ ID NO: 44 - protein, 43 aa, Artificial Sequence
  Synthesized Peptide
CAAELAALEAELAALEGEVNMTRARAGKLLVLKVKLHGLKQAC

SEQ ID NO: 45 - protein, 43 aa, Artificial Sequence
  Synthesized Peptide
CAAELAALEAELAALEVGRVKTLGRYGKLMVLKHQLRNLKCAC

SEQ ID NO: 46 - protein, 43 aa, Artificial Sequence
  Synthesized Peptide
CAAELAALEAELAALEGLDYHTVLSTGKLMVLKRKLNILKLAC

SEQ ID NO: 47 - protein, 43 aa, Artificial Sequence
  Synthesized Peptide
CAAELAALEAELAALEGPPRPHLPPLGKLFLLKFKLYLLKGAC

SEQ ID NO: 48 - protein, 43 aa, Artificial Sequence
  Synthesized Peptide
CAAELAALEAELAALEGPSRVGSRRRGKLNLLKYKLHGLKLAC

SEQ ID NO: 49 - protein, 9 aa, Artificial Sequence
  Synthesized Peptide
WQPPRARIG

SEQ ID NO: 50 - protein, 17 aa, Artificial Sequence
  Synthesized Peptide
CAAELAALEAELAALEG

SEQ ID NO: 51 - protein, 35 aa, Artificial Sequence
  Synthesized Peptide
AELAALEAELAALEGGGGGGGKLAALKAKLAALKA

(19) United States
(12) Patent Application Publication
(10) Pub. No.: US 2023/0139883 A1
(43) Pub. Date: May 4, 2023

(54) Peptides for Inducing Differentiation

(71) Applicants: Shimadzu Corporation, Kyoto-shi, Kyoto (JP); University Public Corporation Osaka, Osaka-shi, Osaka (JP)

(72) Inventors: Hayato Matsui, Kyoto-shi, Kyoto (JP); Junko Enomoto, Kyoto-shi, Kyoto (JP); Masataka Michigami, Sakai-shi, Osaka (JP); Ikuo Fujii, Sakai-shi, Osaka (JP)

(73) Assignees: Shimadzu Corporation, Kyoto-shi, Kyoto (JP); University Public Corporation Osaka, Osaka-shi, Osaka (JP)

(21) Appl. No.: 17/918,105

(22) PCT Filed: Apr. 12, 2021
(86) PCT No.: PCT/JP2021/015186

§ 371 Date: Oct. 10, 2022

(30) Foreign Application Priority Data
Apr. 13, 2020 (JP) 2020-071429

(51) Int. Cl. C07K 14/00; C12N 5/07

(52) U.S. Cl. CPC C07K 14/00; C12N 5/06; C12N 2500/00; C12N 2501/16; C12N 2501/115; C12N 2501/155; C12N 2501/165; C12N 2501/33

(57) ABSTRACT

The present invention chiefly aims to provide a new differentiation inducing agent (peptide) that can solve the previous problems in inducing differentiation of pluripotent stem cells such as ES/iPS cells into somatic cells (e.g., cardiomyocytes).

The present invention can include, for example, a synthetic peptide having, in the molecular structure thereof, an amino acid sequence represented by the following (SEQ ID NO: 1), or an amino acid sequence formed by substitution, deletion and/or addition of one or several amino acid residues in the amino acid sequence. It can also include, for example, a composition for inducing differentiation of pluripotent stem cells into somatic cells, comprising said synthetic peptide.

(SEQ ID No. 1)
C A X X L X X L X X X L X X L X G X X X X X X X X X X X X X X L X X X L X X L X X A C
• • •

### CROSS-REFERENCE TO RELATED APPLICATIONS

This application claims the benefit of Japanese Patent Application No. 2020-71429, filed with the Japan Patent Office on Apr. 13, 2020. The Japanese application is hereby incorporated by reference for all purposes as if the entire application documents (specification, claims, drawings, and abstract) were expressly set forth herein.

### TECHNICAL FIELD

The present invention belongs to the technical field of cell culture including regenerative medicine. The present invention relates to a liquid factor for inducing differentiation from pluripotent stem cells to somatic cells. In detail, the present invention relates to a peptide useful for inducing differentiation from pluripotent stem cells to somatic cells.

### BACKGROUND OF THE INVENTION

When cells are transplanted for the treatment of organ failure, etc., the shortage of donors makes it difficult to provide on-demand medical care to patients in need. Therefore, regenerative medicine, in which necessary organs are produced ex vivo from pluripotent stem cells such as ES/iPS cells and applied to patients, has been attracting attention. In order to produce necessary organs, it is common to apply liquid factors such as growth factors and differentiation inducing factors to cells at appropriate timings, determining sequentially the directions of cell differentiation.

However, the high cost of the liquid factors used increases the total cost of producing the desired cells, which is a barrier to the spread of regenerative medicine. Another problem is that the functions of the produced cells are not as good as those of cells in vivo. The reason for the formerly mentioned high cost is that the liquid factors are mainly composed of proteins made from animal cells, etc. One possible reason for the latterly mentioned lack of function is that the in vivo organogenesis is not sufficiently mimicked in vitro, i.e., the combination of liquid factors to be added is not sufficient.

To solve the former problem, methods to replace proteins with low molecular weight compounds have been attempted. However, considering that the mechanism of action is often unknown, there may be unpredictable risks such as side effects (e.g., infection risks (viruses, mycoplasma, prions, etc.)). To solve the latter problem, it is effective to increase the concentration of liquid factors added, to increase the number of types, and to improve the structure of liquid factors. However, they may cause further cost increase and complication of work.

The following specific technologies, for example, are known to produce necessary organs in vitro from pluripotent stem cells such as ES/iPS cells.

The invention described in Patent Document 1 relates to a method for producing cardiomyocytes from pluripotent stem cells, and it is possible to efficiently induce differentiation into myocardium by using liquid factors such as activin, BMP4, bFGF, VEGF, etc. However, the method is costly because it uses multiple liquid factors.

The inventions described in Patent Documents 2 to 5 relate to a method of inducing differentiation into myocardium using a low molecular weight compound. The invention of Patent Document 2 uses feeder cells, OP9, and requires separation of cells using a cell sorter, which is a complicated process. The inventions in Patent Documents 3, 4, and 5 are specialized for cardiomyocytes, and it is unclear whether they can be used for other differentiation lines.

The invention described in Patent Document 6 induces differentiation of cells by a peptide containing a membrane-permeating peptide and a partial peptide of ephrin. However, since the differentiation induction by said peptide acts to promote differentiation into the hepatocyte lineage, said peptide is not likely to be used in other differentiation inductions.

The invention described in Patent Document 7 relates to a method of inducing differentiation of pluripotent stem cells into somatic cells by using a medium containing heparin-binding growth factor. The method of inducing differentiation is characterized in that the cells are brought into contact with a conjugate comprising a laminin E8 fragment and a fragment containing a growth factor binding moiety of heparan sulfate proteoglycan. The invention enables highly efficient induction of differentiation from pluripotent stem cells to any somatic cells, but the cost is high because animal cells are used for production.

### PRIOR ART

### Patent Document

Patent Document 1: JP, 6429280, B

Patent Document 2: JP, 5611035, B

Patent Document 3: WO 2012/026491

Patent Document 4: WO 2015/182765

Patent Document 5: WO 2015/037706

Patent document 6: JP, 2011-98900, A

Patent Document 7: WO 2018/088501

### SUMMARY OF THE INVENTION

### Problem to be Solved by the Invention

In order to achieve highly efficient differentiation induction of cells, development of methods to induce differentiation from pluripotent stem cells such as ES/iPS cells has been actively pursued. On the other hand, most of such systems are accompanied by one or more of the following problems.
Use of multiple liquid factors.
Expensive due to the use of proteins for the liquid factors.
Proteins are made using cells, so it is difficult to specify (standardize) their contents.
The function of the cardiomyocytes produced, for example, is not sufficient.

The present invention chiefly aims to provide a new differentiation inducing agent or liquid factor (peptide) that can solve the above problems in inducing differentiation of pluripotent stem cells such as ES/iPS cells into somatic cells (e.g., cardiomyocytes). The subject of the present invention may also include, for example, providing a new method for inducing differentiation from pluripotent stem cells to somatic cells and a new method for producing somatic cells.

### Means for Solving the Problem

The inventors have studied intensively and found that the differentiation induction of pluripotent stem cells into somatic cells is enabled by the application of a FGF (Fibroblast Growth Factor) receptor-binding peptide as liquid factors together with activin at post day 1 of the process of inducing differentiation, whereby the above-mentioned problems were solved, and completed the present invention.

The present invention can include embodiments as follows.

[1] A synthetic peptide having, in the molecular structure thereof, an amino acid sequence represented by the following (SEQ ID NO: 1), or an amino acid sequence formed by substitution, deletion and/or addition of one or several amino acid residues in the amino acid sequence:

[Chem 1]
(SEQ ID No. 1)
C A X X L X X L X X X L X X L X G X X X X X X X X X X X X X X L X X X L X X L X X A C
• • •

in the formula, the 1st X from the N-terminus of SEQ ID NO: 1 is A, R, Q, V, L, K, F, or H, the 2nd X is E or H, the 3rd X is A, Q, V, Y, R, L, or E, the 4th X is A or S, the 5th X is E, L, H, R, G, or K, the 6th X is A, K, M, R, G, or Y, the 7th X is E or D, the 8th X is A, Q, R, Y, E, K, or G, the 9th X is A or I, the 10th X is E, G, Y, Q, R, K, or M, the 11th X is F, A, Q, V, G, I, N, R, M, D, L, S, P, K, or E, the 12th X is G, V, K, T, P, R, M, N, E, D, S, C, W, H, or A, the 13th X is G, R, E, F, A, K P, N, L, V, M, D, H, T, or Y, the 14th X is V, D, L, M, S, T, A, N, H, G, F, E, R, P, Y, or K, the 15th X is V, K, Y, T, N, G, D, E, P, F, Q, H, or I, the 16th X is Y, R, H, L, P, N, E, M, A, G, V, I, or S, the 17th X is S, K, M, G, H, Q, T, V, C, L, N, A, E, P, or R, the 18th X is C, S, A, T, R, E, N, Q, G, K, Y, P, L, V, or M, the 19th X is E, K, S, V, L, R, G, I, F, T, A, H, Q, N, M, or Y, the 20th X is W or G, the 21st X is Q, K, F, or H, the 22nd X is A or L, the 23rd X is V, M, D, Y, K, L, I, R, S, Q, G, E, H, F, N, or A, the 24th X is Y, M, R, S, K, H, G, L, N, E, V, D, or A, the 25th X is H, F, Y, K, I, Q, M, or V, the 26th X is Y, W, G, L, N, D, Q, E, M, F, R, H, S, K, V, or I, the 27th X is K, R, or Q, the 28th X is M, E, W, V, H, S, N, I, Q, Y, G, L, R, F, or A, the 29th X is R, L, V, S, H, Y, F, N, K, Q, W, E, I, G, or A, the 30th X is F, R, D, Q, M, K, L, or H, and the 31st X is Q, G, Y, R, N, I, S, H, E, M, L, D, C, W, V, or F.
[2] The synthetic peptide according to the [1] above, having, in the molecular structure thereof, an amino acid sequence represented by the following (SEQ ID NO: 2), or an amino acid sequence formed by substitution, deletion and/or addition of one or several amino acid residues in the amino acid sequence:

[Chem 2]
(SEQ ID No. 2)
C A A E L A A L E A E L A A L E G X X X X X X X X X X X X X X L K X X L X X L K X A C
• • •

in the formula, the 1st X from the N-terminus of SEQ ID NO: 2 is F, A, Q, V, G, I, N, R, M, D, L, S, P, K, or E, the 2nd X is G, V, K, T, P, R, M, N, E, D, S, C, W, H, or A, the 3rd X is G, R, E, F, A, K, P, N, L, V, M, D, H, T, or Y, the 4th X is V, D, L, M, S, T, A, N, H, G, F, E, R, P, Y, or K, the 5th X is V, K, Y, T, N, G, D, E, P, F, Q, H, or I, the 6th X is Y, R, H, L, P, N, E, M, A, G, V, or I, the 7th X is S, K, M, G, H, Q, T, V, C, L, N, A, E, or R, the 8th X is C, S, A, T, R, E, N, Q, G, K, Y, P, L, V, or M, the 9th X is E, K, S, V, L, R, G, I, F, T, A, H, Q, N, M, or Y, the 10th X is W or G, the 11th X is Q or K, the 12th X is A or L, the 13th X is V, M, D, Y, K, L, I, R, S Q, G, or E, the 14th X is Y, M, R, S, K, H, G, L, N, E, or V, the 15th X is Y, W, G, L, N, D, Q, E, M, F, R, H, S, K, or V, the 16th X is K or R, the 17th X is M, E, W, V, H, S, N, I, Q, Y, G, L, or R, the 18th X is R, L, V, S, H, Y, F, N, K, Q, W, E, I, or G, and the 19th X is Q, G, Y, R, N, I, S, H, E, M, L, D, C, W, or V.
[3] The synthetic peptide according to the [2] above, wherein the 1st X from the N-terminus of said amino acid sequence (SEQ ID NO: 2) is F, A, Q, V, G, I, N, R, M, D, L, S, or P, the 2nd X is G, V, K, T, P, R, M, N, E, D, S, C, W, or H, the 3rd X is G, R, E, F, A, K, P, N, L, V, or M, the 4th X is V, D, L, M, S, T, A, N, H, G, F, E, or R, the 5th X is V, K, Y, T, N, G, D, E, P, F, Q, or H, the 6th X is Y, R, H, L, P, N, E, M, A, G, or V, the 7th X is S, K, M, G, H, Q, T, V, C, L, N, or A, the 8th X is C, S, A, T, R, E, N, Q, G, K, Y, or P, the 9th X is E, K, S, V, L, R, G, I, F, T, A, or H, the 10th X is W or G, the 13st X is V, M, D, Y, K, L, I, or R, the 14th X is Y, M, R, S, K, H, G, L, N, or E, the 15th X is Y, W, G, L, N, D, Q, E, M, F, or R, the 16th X is K, the 17th X is M, E, W, V, H, S, N, I, Q, or Y, the 18th X is R, L, V, S, H, Y, F, N, or K, and the 19th X is Q, G, Y, R, N, I, S, H, E, M, L, or D.
[4] The synthetic peptide according to the [3] above, wherein said amino acid sequence (SEQ ID NO: 1) is any amino acid sequence selected from the group consisting of SEQ ID Nos: 4 to 26.
[5] The synthetic peptide according to the [2] above, wherein the 1st X from the N-terminus of said amino acid sequence (SEQ ID NO: 2) is A, Q, V, G, R, L, S, K, or E, the 2nd X is G, V, K, T, P, R, N, E, D, S, H, or A, the 3rd X is E, F, A, P, N, V, M, D, H, T, or Y, the 4th X is V, L, M, S, T, H, F, E, R, P, Y, or K, the 5th X is V, T, or I, the 6th X is R, L, M, G, V, or I, the 7th X is S, M, G, T, L, N, A, E, or R, the 8th X is S, T, R, Q, G, L, V, or M, the 9th X is K, S, V, L, R, T, A, Q, N, M, or Y, the 10th X is G, the 11th X is K, the 12th X is L, the 13th X is V, M, L, I, S, Q, G, or E, the 14th X is M, S, H, G, L, or V, and the 15th X is Y, L, Q, E, M, F, R, H, S, K, or V, the 17th X is E, W, V, H, N, Q, G, L, or R, the 18th X is R, S, H, N, Q, W, E, I, or G, and the 19th X is Q, G, R, H, E, L, D, C, W, or V.
[6] The synthetic peptide according to the [5] above, wherein said amino acid sequence (SEQ ID NO: 1) is any amino acid sequence selected from the group consisting of SEQ ID Nos: 27 to 46.
[7] The synthetic peptide according to the [1] above, having, in the molecular structure thereof, an amino acid sequence represented by the following amino acid sequence (SEQ ID NO: 3), or an amino acid sequence formed by substitution, deletion and/or addition of one or several amino acid residues in the amino acid sequence:

[Chem 3]
(SEQ ID No. 3)
C A X X L X X L X X X L X X L X G X X X X X X X X
X X X L X X L X X X L X X L X X A C
• • •

in the formula, the 1st X from the N-terminus of SEQ ID NO: 3 is A, R, Q, V, L, K, F, or H, the 2nd X is E or H, the 3rd X is A, Q, V, Y, R, L, or E, the 4th X is A or S, the 5th X is E, L, H, R, G, or K, the 6th X is A, K, M, R, G, or Y, the 7th X is E or D, the 8th X is A, Q, R, Y, E, K, or G, the 9th X is A or I, the 10th X is E, G, Y, Q, R, K, or M, the 11th X is G or P, the 12th X is G, P, D, or S, the 13th X is G or R, the 14th X is V, G, or P, the 15th X is G or H, the 16th X is L, G, or S, the 17th X is P or R, the 18th X is R or P, the 19th X is L or R, the 20th X is G, the 21st X is K, F, or H, the 22nd X is M, Y, L, R, Q, G, H, F, N, or A, the 23rd X is R, L, N, D, or A, the 24th X is H, F, Y, K, Q, M, or V, the 25th X is Y, L, F, R, V, or I, the 26th X is K or Q, the 27th X is H, Y, L, F, or A, the 28th X is L, N, E, G, or A, the 29th X is F, R, D, Q, M, K, L, or H, and the 30th X is G, N, I, H, L, W, V, or F.
[8] The synthetic peptide according to the [7] above, wherein the 17th and 18th Xs from the N-terminus of said amino acid sequence (SEQ ID NO: 1) are P and the 19th X is L, or all of the 17th to 19th Xs are R.
[9] The synthetic peptide according to the [7] or [8] above, wherein said SEQ ID NO: 1 is the amino acid sequence of SEQ ID NO: 47 or 48.
[10] The synthetic peptide according to any one of the [1] to [9] above, comprising any amino acid sequence selected from the group consisting of SEQ ID Nos: 4 to 48, or an amino acid sequence formed by substitution, deletion and/or addition of one or several amino acid residues in the amino acid sequence.
[11] The synthetic peptide according to any one of the [1] to [10] above, wherein WQPPRARIG (SEQ ID NO: 49) is bound through a linker or directly.
[12] The synthetic peptide according to any one of the [1] to [11] above, wherein said synthetic peptide is dimerized.
[13] The synthetic peptide according to any one of the [1] to [12] above, wherein the end of said synthetic peptide is molecularly modified.
[14] A composition for inducing differentiation of a pluripotent stem cell into a somatic cell, comprising the synthetic peptide according to any one of the [1] to [13] above.
[15] The composition according to the [14] above, further comprising one or more selected from the group consisting of activin, bFGF, BMP4, VEGF, IWP-3, transferrin, and an extracellular matrix.
[16] The composition according to the [14] or [15] above, wherein said somatic cell is an ectodermal, mesodermal, or endodermal cell.
[17] The composition according to the [16] above, wherein said ectodermal cell is a neuron, said mesodermal cell is a cardiomyocyte, and said endodermal cell is a hepatocyte.
[18] The composition according to any one of the [14] to [17] above, which is a culture medium or a culture substrate.
[19] A process for producing a somatic cell, comprising a step of applying a liquid factor containing a synthetic peptide having binding properties to the FGF receptor to the embryoid body of a pluripotent stem cell.
[20] The process for producing a somatic cell according to the [19] above, wherein the liquid factor further comprises another liquid factor for inducing differentiation.
[21] The process for producing a somatic cell according to the [19] or [20] above, wherein the peptide is one or more synthetic peptides according to any one of the [1] to [13] above.
[22] The process for producing a somatic cell according to the [20] or [21] above, wherein said another liquid factor for inducing differentiation is one or more selected from the group consisting of activin, bFGF, BMP4, VEGF, IWP-3, and transferrin.
[23] The process for producing a somatic cell according to any one of the [19] to [22] above, wherein the somatic cell is an ectodermal, mesodermal, or endodermal cell.
[24] The process for producing a somatic cell according to the [23] above, wherein said ectodermal cell is a neuron, said mesodermal cell is a cardiomyocyte, and said endodermal cell is a hepatocyte.
[25] A method of inducing differentiation of a pluripotent stem cell into a somatic cell, comprising a step of applying a liquid factor containing a synthetic peptide having binding properties to the FGF receptor to the embryoid body of a pluripotent stem cell.
[26] The method of inducing differentiation according to the [25] above, wherein the liquid factor further comprises another liquid factor for inducing differentiation.
[27] The method of inducing differentiation according to the [25] or [26] above, wherein the peptide is one or more synthetic peptides according to any one of the [1] to [13] above.
[28] The method of inducing differentiation according to the [26] or [27] above, wherein said another liquid factor for inducing differentiation is one or more selected from the group consisting of activin, bFGF, BMP4, VEGF, IWP-3, and transferrin.
[29] The method of inducing differentiation according to any one of the [25] to [28] above, wherein the somatic cell is an ectodermal, mesodermal, or endodermal cell.
[30] The method of inducing differentiation according to the [29] above, wherein said ectodermal cell is a neuron, said mesodermal cell is a cardiomyocyte, and said endodermal cell is a hepatocyte.

### EFFECT OF THE INVENTION

The present invention can efficiently induce differentiation from pluripotent stem cells to somatic cells using synthetic peptides of medium molecular weight, thus saving costs and other expenses compared to conventional methods. In addition, when used in combination with other liquid factors for inducing differentiation, such as bFGF, the said induction of differentiation can be promoted more efficiently.

### BRIEF DESCRIPTION OF THE DRAWING

FIG. 1 represents protocols for induction of cardiomyocyte differentiation.

FIG. 2 represents three-dimensional structures of the phage surface-displayed peptide library.

FIG. 3 shows a flowchart from acquiring an FGFR1-binding phage from the phage library eluted after panning, up to comprehensively analyzing peptide library-derived sequences contained in the phagemid DNA.

FIG. 4 represents results of experiments on binding to FGFR by surface plasmon resonance method. The left end represents the results for FGF receptor 1, the middle represents the results for FGF receptor 2, and the right end represents the results for FGF receptor 3. In each figure, the vertical axis represents response units (RU), and the horizontal axis represents time (seconds).

FIG. 5 shows expression levels of cardiac gene markers. The left figure shows cTNT gene expression levels, and the right figure shows Actinin-2 gene expression levels.

FIG. 6 is photographs of immunostained cells.

FIG. 7 is photographs of immunostained cells.

FIG. 8 represents expression levels of cardiac gene markers. The left figure shows cTNT gene expression levels, and the right figure shows Actinin-2 gene expression levels.

FIG. 9 represents expression levels of cardiac gene markers. The left figure shows cTNT gene expression levels, and the right figure shows Actinin-2 gene expression levels.

FIG. 10 represents expression levels of cardiac gene markers. The left figure shows cTNT gene expression levels, and the right figure shows Actinin-2 gene expression levels.

FIG. 11 represents results of experiments on binding to FGFR of peptides HBD-YX and HBD-F3-100nX by surface plasmon resonance method.

FIG. 12 represents protocols for induction of myocardial differentiation.

FIG. 13 represents results of gene expression analysis in induction of myocardial differentiation.

FIG. 14 represents protocols for induction of hepatic differentiation.

FIG. 15 represents results of gene expression analysis in hepatic differentiation.

FIG. 16 represents results of CYP3A4 activity measurement in hepatic differentiation.

FIG. 17 represents protocols for induction of neural differentiation.

FIG. 18 represents results of gene expression analysis in neural differentiation.

### EMBODIMENT FOR CARRYING OUT THE PRESENT INVENTION

The present invention is described in detail below.

Matters other than those specifically mentioned herein (e.g., primary structure and chain length of peptides) that are necessary for the practice of the invention (e.g., general matters such as peptide synthesis, cell culture techniques, and preparation of pharmaceutical compositions with peptides as ingredients) may be understood as matters of design of those skilled in the art based on conventional techniques in the fields of cell engineering, medicine, pharmaceutical sciences, organic chemistry, biochemistry, genetic engineering, protein engineering, molecular biology, hygiene, etc. The present invention can be implemented based on the contents disclosed herein and the common technical knowledge in the field. In the following description, amino acids, as the case may be, are represented by using one-letter notation (but three-letter notation in the sequence listing) in accordance with the nomenclature for amino acids given in the IUPAC-IUB guidelines. Amino acid sequences described herein are always N-terminal on the left side and C-terminal on the right side.

The entire contents of all references cited herein are incorporated herein by reference.

### 1 Synthetic Peptides for the Present Invention

The synthetic peptide of the present invention (hereinafter referred to as the “present inventive peptide”) is characterized by having, in the molecular structure thereof, an amino acid sequence (SEQ ID NO: 1) represented by the following, or an amino acid sequence formed by substitution, deletion and/or addition of one or several amino acid residues in the amino acid sequence. In the present invention, “several” means a small number of substitutions, deletions and/or additions of amino acid residues to the extent that the effect of the invention is not impaired even if such substitutions, deletions and/or additions occur, and usually means about 12 or less. It is preferred that the number of amino acid residues to be substituted, deleted and/or added is no more than 10. More preferably, the number is 5 or less, 4 or less, 3 or less, or 2 or less.

[Chem 4]
(SEQ ID No. 1)
C A X X L X X L X X X L X X L X G X X X X X X X X
X X X X X X L X X X L X X L X X A C
• • •

(In the formula, the 1st X from the N-terminus of SEQ ID NO: 1 is A, R, Q, V, L, K, F, or H, the 2nd X is E or H, the 3rd X is A, Q, V, Y, R, L, or E, the 4th X is A or S, the 5th X is E, L, H, R, G, or K, the 6th X is A, K, M, R, G, or Y, the 7th X is E or D, the 8th X is A, Q, R, Y, E, K, or G, the 9th X is A or I, the 10th X is E, G, Y, Q, R, K, or M, the 11th X is F, A, Q, V, G, I, N, R, M, D, L, S, P, K, or E, the 12th X is G, V, K, T, P, R, M, N, E, D, S, C, W, H, or A, the 13th X is G, R, E, F, A, K P, N, L, V, M, D, H, T, or Y, the 14th X is V, D, L, M, S, T, A, N, H, G, F, E, R, P, Y, or K, the 15th X is V, K, Y, T, N, G, D, E, P, F, Q, H, or I, the 16th X is Y, R, H, L, P, N, E, M, A, G, V, I, or S, the 17th X is S, K, M, G, H, Q, T, V, C, L, N, A, E, P, or R, the 18th X is C, S, A, T, R, E, N, Q, G, K, Y, P, L, V, or M, the 19th X is E, K, S, V, L, R, G, I, F, T, A, H, Q, N, M, or Y, the 20th X is W or G, the 21st X is Q, K, F, or H, the 22nd X is A or L, the 23rd X is V, M, D, Y, K, L, I, R, S, Q, G, E, H, F, N, or A, the 24th X is Y, M, R, S, K, H, G, L, N, E, V, D, or A, the 25th X is H, F, Y, K, I, Q, M, or V, the 26th X is Y, W, G, L, N, D, Q, E, M, F, R, H, S, K, V, or I, the 27th X is K, R, or Q, the 28th X is M, E, W, V, H, S, N, I, Q, Y, G, L, R, F, or A, the 29th X is R, L, V, S, H, Y, F, N, K, Q, W, E, I, G, or A, the 30th X is F, R, D, Q, M, K, L, or H, and the 31st X is Q, G, Y, R, N, I, S, H, E, M, L, D, C, W, V, or F.)

The present inventive peptide may be an embodiment having, in the molecular structure thereof, an amino acid sequence of said SEQ ID NO: 1 in which the 1st X from the N-terminus is A, the 2nd X is E, the 3rd X is A, the 4th X is A, the 5th X is E, the 6th X is A, the 7th X is E, the 8th X is A, the 9th X is A, the 10th X is E, the 16th X is Y, R, H, L, P, N, E, M, A, G, V, or I, the 17th X is S, K, M, G, H, Q, T, V, C, L, N, A, E, or R, the 21st X is Q or K, the 23rd X is V, M, D, Y, K, L, I, R, S, Q, G, or E, the 24th X is Y, M, R, S, K, H, G, L, N, E, or V, the 25th X is K, the 26th X is Y, W, G, L, N, D, Q, E, M, F, R, H, S, K, or V, the 27th X is K or R, the 28th X is M, E, W, V, H, S, N, I, Q, Y, G, L, or R, the 29th X is R, L, V, S, H, Y, F, N, K, Q, W, E, I, or G, the 30th X is K, and the 31st X is Q, G, Y, R, N, I, S, H, E, M, L, D, C, W, or V; or may be an embodiment having, in the molecular structure thereof, an amino acid sequence formed by substitution, deletion and/or addition of one or several amino acid residues in the amino acid sequence (hereinafter also referred to as “100nX type peptides”).

That is, the 100nX type peptides have, in the molecular structure thereof, the amino acid sequence (SEQ ID NO: 2) represented by the following, or an amino acid sequence formed by substitution, deletion and/or addition of one or several amino acid residues in the amino acid sequence.

[Chem 5]
(SEQ ID No. 2)
C A A E L A A L E A E L A A L E G X X X X X X X X
X X X X X X L K X X L X X L K X A C
• • •

(In the formula, the 1st X from the N-terminus of SEQ ID NO: 2 is F, A, Q, V, G, I, N, R, M, D, L, S, P, K, or E, the 2nd X is G, V, K, T, P, R, M, N, E, D, S, C, W, H, or A, the 3rd X is G, R, E, F, A, K, P, N, L, V, M, D, H, T, or Y, the 4th X is V, D, L, M, S, T, A, N, H, G, F, E, R, P, Y, or K, the 5th X is V, K, Y, T, N, G, D, E, P, F, Q, H, or I, the 6th X is Y, R, H, L, P, N, E, M, A, G, V, or I, the 7th X is S, K, M, G, H, Q, T, V, C, L, N, A, E, or R, the 8th X is C, S, A, T, R, E, N, Q, G, K, Y, P, L, V, or M, the 9th X is E, K, S, V, L, R, G, I, F, T, A, H, Q, N, M, or Y, the 10th X is W or G, the 11th X is Q or K, the 12th X is A or L, the 13th X is V, M, D, Y, K, L, I, R, S, Q, G, or E, the 14th X is Y, M, R, S, K, H, G, L, N, E, or V, the 15th X is Y, W, G, L, N, D, Q, E, M, F, R, H, S, K, or V, the 16th X is K or R, the 17th X is M, E, W, V, H, S, N, I, Q, Y, G, L, or R, the 18th X is R, L, V, S, H, Y, F, N, K, Q, W, E, I, or G, and the 19th X is Q, G, Y, R, N, I, S, H, E, M, L, D, C, W, or V.)

The 100nX type peptides may be an embodiment having, in the molecular structure thereof, an amino acid sequence of said SEQ ID NO: 1 in which, further, the 11th X from the N-terminus is F, A, Q, V, G, I, N, R, M, D, L, S, or P, the 12th X is G, V, K, T, P, R, M, N, E, D, S, C, W, or H, the 13th X is G, R, E, F, A, K, P, N, L, V, or M, the 14th X is V, D, L, M, S, T, A, N, H, G, F, E, or R, the 15th X is V, K, Y, T, N, G, D, E, P, F, Q, or H, the 16th X is Y, R, H, L, P, N, E, M, A, G, or V, the 17th X is S, K, M, G, H, Q, T, V, C, L, N, or A, the 18th X is C, S, A, T, R, E, N, Q, G, K, Y, or P, the 19th X is E, K, S, V, L, R, G, I, F, T, A, or H, the 20th X is W or G, the 23rd X is V, M, D, Y, K, L, I, or R, the 24th X is Y, M, R, S, K, H, G, L, N, or E, the 26th X is Y, W, G, L, N, D, Q, E, M, F, or R, the 27th X is K, the 28th X is M, E, W, V, H, S, N, I, Q, or Y, the 29th X is R, L, V, S, H, Y, F, N, or K, and the 31st X is Q, G, Y, R, N, I, S, H, E, M, L, or D; or may be an embodiment having, in the molecular structure thereof, an amino acid sequence formed by substitution, deletion and/or addition of one or several amino acid residues of the amino acid sequence (hereinafter also referred to as “100nX” or “100nX-Monomer”).

That is, 100nX has, in the molecular structure thereof, an amino acid sequence of said SEQ ID NO: 2 in which, further, the 1st X from the N-terminus is F, A, Q, V, G, I, N, R, M, D, L, S, or P, the 2nd X is G, V, K, T, P, R, M, N, E, D, S, C, W, or H, the 3rd X is G, R, E, F, A, K, P, N, L, V, or M, the 4th X is V, D, L, M, S, T, A, N, H, G, F, E, or R, the 5th X is V, K, Y, T, N, G, D, E, P, G, F, Q, or H, the 6th X is Y, R, H, L, P, N, E, M, A, G, or V, the 7th X is S, K, M, G, H, Q, T, V, C, L, N, or A, the 8th X is C, S, A, T, R, E, N, Q, G, K, Y, or P, the 9th X is E, K, S, V, L, R, G, I, F, T, A, or H, the 10th X is W or G, the 13th X is V, M, D, Y, K, L, I, or R, the 14th X is Y, M, R, S, K, H, G, L, N, or E, the 15th X is Y, W, G, L, N, D, Q, E, M, F, or R, the 16th X is K, the 17th X is M, E, W, V, H, S, N, I, Q, or Y, the 18th X is R, L, V, S, H, Y, F, N, or K, and the 19th X is Q, G, Y, R, N, I, S, H, E, M, L, or D; or has, in the molecular structure thereof, an amino acid sequence formed by substitution, deletion and/or addition of one or several amino acid residues in the amino acid sequence.

In addition, the 100nX type peptides may be an embodiment having, in the molecular structure thereof, an amino acid sequence of said SEQ ID NO: 1 in which, further, the 11th X from the N-terminus is A, Q, V, G, R, L, S, K, or E, the 12th X is G, V, K, T, P, R, N, E, D, S, H, or A, the 13th X is E, F, A, P, N, V, M, D, H, T, or Y, the 14th X is V, L, M, S, T, H, F, E, R, P, Y, or K, the 15th X is V, T, or I, the 16th X is R, L, M, G, V, or I, the 17th X is S, M, G, T, L, N, A, E, or R, the 18th X is S, T, R, Q, G, L, V, or M, the 19th X is K, S, V, L, R, T, A, Q, N, M, or Y, the 20th X is G, the 21st X is K, the 22nd X is L, the 23rd X is V, M, L, I, S, Q, G, or E, the 24th X is M, S, H, G, L, or V, the 26th X is Y, L, Q, E, M, F, R, H, S, K, or V, the 28th X is E, W, V, H, N, Q, G, L, or R, the 29th X is R, S, H, N, Q, W, E, I, or G, and the 31st X is Q, G, R, H, E, L, D, C, W, or V; or may be an embodiment having, in the molecular structure thereof, an amino acid sequence formed by substitution, deletion and/or addition of one or several amino acid residues in the amino acid sequence (hereinafter also referred to as “F3-100nX” or “F3-100nX-Monomer”).

That is, F3-100nX has, in the molecular structure thereof, an amino acid sequence of said SEQ ID NO: 2 in which, further, the 1st X from the N-terminus is A, Q, V, G, R, L, S, K, or E, the 2nd X is G, V, K, T, P, R, N, E, D, S, H, or A, the 3rd X is E, F, A, P, N, V, M, D, H, T, or Y, the 4th X is V, L, M, S, T, H, F, E, R, P, Y, or K, the 5th X is V, T, or I, the 6th X is R, L, M, G, V, or I, the 7th X is S, M, G, T, L, N, A, E, or R, the 8th X is S, T, R, Q, G, L, V, or M, the 9th X is K, S, V, L, R, T, A, Q, N, M, or Y, the 10th X is G, the 11th X is K, the 12th X is L, the 13th X is V, M, L, I, S, Q, G, or E, the 14th X is M, S, H, G, L, or V, the 15th X is Y, L, Q, E, M, F, R, H, S, K, or V, the 17th X is E, W, V, H, N, Q, G, L, or R, the 18th X or R, S, H, N, Q, W, E, I, or G, and the 19th X is Q, G, R, H, E, L, D, C, W, or V; or has, in the molecular structure thereof, an amino acid sequence formed by substitution, deletion and/or addition of one or several amino acid residues in the amino acid sequence.

On the other hand, the present inventive peptide may be an embodiment having, in the molecular structure thereof, an amino acid sequence of said SEQ ID NO: 1 in which the 11th X from the N-terminus is G or P, the 12th X is G, P, D, or S, the 13th is G or R, the 14th X is V, G, or P, the 15th X is G or H, the 16th X is L, G, or S, the 17th X is P or R, the 18th X is R or P, the 19th X is L or R, the 20th X is G, the 21st X is K, F, or H, the 22nd X is L, the 23rd X is M, Y, L, R, Q, G, H, F, N, or A, the 24th X is R, L, N, D, or A, the 26th X is Y, L, F, R, V, or I, the 27th X is K or Q, the 28th X is H, Y, L, F, or A, the 29th X is L, N, E, G, or A, and the 31st X is G, N, I, H, L, W, V, or F; or may be an embodiment having, in the molecular structure thereof, an amino acid sequence formed by substitution, deletion and/or addition of one or more amino acid residues in the amino acid sequence (hereinafter also referred to as “YX type peptides”).

That is, the YX type peptides have, in the molecular structure thereof, the amino acid sequence (SEQ ID NO: 3) represented by the following, or an amino acid sequence formed by substitution, deletion and/or addition of one or several amino acid residues in the amino acid sequence.

[Chem 6]
(SEQ ID No. 3)
C A X X L X X L X X X L X X L X G X X X X X X X X
X X X L X X L X X X L X X L X X A C
• • •

(In the formula, the 1st X from the N-terminus of SEQ ID NO: 3 is A, R, Q, V, L, K, F, or H, the 2nd X is E or H, the 3rd X is A, Q, V, Y, R, L, or E, the 4th X is A or S, the 5th X is E, L, H, R, G, or K, the 6th X is A, K, M, R, G, or Y, the 7th X is E or D, the 8th X is A, Q, R, Y, E, K, or G, the 9th X is A or I, the 10th X is E, G, Y, Q, R, K, or M, the 11th X is G or P, the 12th X is G, P, D, or S, the 13th X is G or R, the 14th X is V, G, or P, 15th X is G or H, the 16th X is L, G, or S, the 17th X is P or R, the 18th X is R or P, the 19th X is L or R, the 20th X is G, the 21st X is K, F, or H, the 22nd X is M, Y, L, R, Q, G, H, F, N, or A, the 23rd X is R, L, N, D, or A, the 24th X is H, F, Y, K, Q, M, or V, the 25th X is Y, L, F, R, V, or I, the 26th X is K or Q, the 27th X is H, Y, L, F, or A, the 28th X is L, N, E, G, or A, the 29th X is F, R, D, Q, M, K, L, or H, and the 30th X is G, N, I, H, L, W, V, or F.)

The YX type peptides may be an embodiment in which, further, the 17th and 18th Xs from the N-terminus of said amino acid sequence (SEQ ID NO: 1 or 3) is P and the 19th X is L, or all of the 17th to 19th Xs are R (hereinafter also referred to as “YX” or “YX-Monomer”).

The term “synthetic peptide” means a peptide fragment that is produced by artificial chemical synthesis or biosynthesis (i.e., production based on genetic engineering) and can exist stably in a given system (e.g., a composition constituting a cell differentiation inducer), rather than being independently and stably existing in nature as a peptide chain per se.

The term “peptide” refers to an amino acid polymer having multiple peptide bonds. Although the term is not limited by the number of amino acid residues in the peptide chain, it typically means peptides with relatively small molecular weight, such as those with the total number of amino acid residues of approximately no more than 100, preferably no more than 50 (e.g., 30 to 50 or 40 to 50).

The term “amino acid residue” is a term that, except where otherwise noted, refers to each amino acid (—NH—C(R)(H)—CO—) in a peptide chain, including the N-terminal and C-terminal amino acids of the peptide chain.

Note that it is preferred that all amino acid residues of the present inventive peptide are L-type amino acids, but some or all of the amino acid residues may be replaced by D-type amino acids as long as the differentiation-inducing activity is not impaired.

Use of the present inventive peptide that binds to the fibroblast growth factor receptor (FGFR) (100nX, F3-100nX, or YX), or a fused peptide of the present inventive peptide with a peptide that binds to heparan sulfate (hereinafter also referred to as “HBD-100nX”, “HBD-F3-100nX”, or “HBD-YX”), or a dimerized peptide of the present inventive peptide (hereinafter also referred to as “100nX-Dimer”, “F3-100nX-Dimer or “YX-Dimer”) enable inducing differentiation of pluripotent stem cells such as iPS cells into somatic cells such as cardiomyocytes with fewer types of liquid factors than in conventional protocols.

Making 100nX, HBD-100nX, or 100n X-Dimer act in combination with bFGF (basic fibroblast growth factor) can promote differentiation into somatic cells such as cardiomyocytes or promote maturation of somatic cells such as cardiomyocytes.

Use of a fused peptide between a peptide that binds to the FGFR (e.g., 100nX, YX, F3-100nX) and a peptide that binds to heparan sulfate (e.g., HBD-100nX, HBD-YX, HBD-F3-100nX) can induce differentiation of cardiomyocytes from iPS cells without the use of bFGF.

Additionally, use of HBD-100nX can induce differentiation of iPS cells into hepatocytes or neurons without the use of bFGF.

Among 100nX, compounds having on the molecular structure thereof a peptide represented by any one of SEQ ID NOs: 4 to 26 shown in Table 1 below, or the peptides represented by SEQ ID NOs: 4 to 26 are preferred. More preferred present inventive peptides are those having on the molecular structure thereof a peptide represented by SEQ ID NO: 5, SEQ ID NO: 8, or SEQ ID NO: 9; or the peptides represented by SEQ ID NO: 5, SEQ ID NO: 8, or SEQ ID NO: 9.

Among F3-100nX, compounds having on the molecular structure thereof a peptide represented by any one of SEQ ID NOs: 27 to 46 shown in Table 2 below, or the peptides represented by SEQ ID NOs: 27 to 46 are preferred. More preferred present inventive peptides are those having on the molecular structure thereof a peptide represented by SEQ ID NO: 27, SEQ ID NO: 28, SEQ ID NO: 29, SEQ ID NO: 30, SEQ ID NO: 31, or SEQ ID NO: 32; or the peptides represented by SEQ ID NO: 27, SEQ ID NO: 28, SEQ ID NO: 29, SEQ ID NO: 30, SEQ ID NO: 31, or SEQ ID NO: 32.

Among YX, compounds having on the molecular structure thereof a peptide represented by either one of SEQ ID NO: 47 or 48 shown in Table 3 below, or the peptides represented by SEQ ID NO: 47 or 48 are preferred.

In some embodiments of the present inventive peptides, the total number of amino acid residues constituting the peptide is no more than 100 (especially, no more than 50), which makes chemical synthesis easy and inexpensive and easy to handle.

TABLE 1

| SEQ | | |
|---|---|---|
| No. | I D | Sequence |
| 4 | 100n17 | CAAELAALEAELAALEGFGGVVYSCE |
| | | WQAVYLKYKLMRLKQAC |
| 5 | 100n85 | CAAELAALEAELAALEGAVGDKRKSK |
| | | GKLMMLKQKLELLKGAC |
| 6 | 100n82 | CAAELAALEAELAALEGVGRLYHMAS |
| | | GKLDRLKGKLMVLKYAC |
| 7 | 100n83 | CAAELAALEAELAALEGVKELTLGTV |
| | | GKLMSLKYKLWSLKQAC |
| 8 | 100n8 | CAAELAALEAELAALEGQTFMNPHRL |
| | | GKLDKLKLKLVHLKRAC |
| 9 | 100n9 | CAAELAALEAELAALEGVPGDNNGTR |
| | | GKLYHLKNKLHRLKNAC |
| 10 | 100n368 | CAAELAALEAELAALEGGRFSGLQEK |
| | | GKLYGLKNKLHYLKRAC |
| 11 | 100n781 | CAAELAALEAELAALEGGMATDHMNR |
| | | GKLYRLKNKLHFLKIAC |
| 12 | 100n423 | CAAELAALEAELAALEGAKKAEEGQK |
| | | GKLYRLKNKLHYLKSAC |
| 13 | 100n12 | CAAELAALEAELAALEGINAVPMSGR |
| | | GKLYSLKNKLHYLKNAC |
| 14 | 100n606 | CAAELAALEAELAALEGNTANDPTAS |
| | | GKLKYLKYKLSNLKHAC |
| 15 | 100n1793 | CAAELAALEAELAALEGREPSGAMGS |
| | | GKLKRLKYKLQNLKHAC |
| 16 | 100n417 | CAAELAALEAELAALEGGDEVPHVGG |
| | | GKLKMLKYQLNNLKHAC |
| 17 | 100n1074 | CAAELAALEAELAALEGMSNHTLGSI |
| | | GKLMLLKDKLESLKEAC |
| 18 | 100n108 | CAAELAALEAELAALEGDCLAGGCSF |
| | | GKLLNLKQKLIRLKRAC |
| 19 | 100n182 | CAAELAALDAELAALEGARLGKMSKV |
| | | GKLIKLKEKLQNLKMAC |
| 20 | 100n84 | CAAELAALEAELAALEGLGGFFNLRT |
| | | GKLDELKMKLQHLKQAC |
| 21 | 100n51 | CAAELAALEAELAALEGAWVEQVNRR |
| | | GKLDGLKMKLQRLKLAC |
| 22 | 100n36 | CAAELAALEAELAALEGVPALVGQEG |
| | | GKLVLLKWKLWRLKNAC |
| 23 | 100n1314 | CAAELAALEAELAALEGSVALGNKAG |
| | | GKLRLLKLKLELLKDAC |
| 24 | 100n1543 | CAAELAALEAELAALEGSSMDHVAYT |
| | | GKLRMLKFRLELLKGAC |
| 25 | 100n1348 | CAAELAALEAELAALEGADAFTLGSA |
| | | GKLMMLKEKLNHLKHAC |
| 26 | 100n222 | CAAELAALEAELAALEGPHPRHHHPH |
| | | GKLKKLKRKLYKLKQAC |

TABLE 2

| SEQ | | |
|---|---|---|
| No. | I D | Sequence |
| 27 | F3-100n9 | CAAELAALEAELAALEGVRPETMSQV |
| | | GKLLVLKHRLGQLKRAC |
| 28 | F3-100n251 | CAAELAALEAELAALEGGRDRTLELS |
| | | GKLLVLKSKLHWLKHAC |
| 29 | F3-100n131 | CAAELAALEAELAALEGKEHPTLRTS |
| | | GKLLHLKLKLHELKRAC |
| 30 | F3-100n410 | CAAELAALEAELAALEGVKELTLGTV |
| | | GKLMSLKYKLWSLKQAC |
| 31 | F3-100n228 | CAAELAALEAELAALEGEGMSTLNRQ |
| | | GKLSVLKSKLLWLKHAC |
| 32 | F3-100n116 | CAAELAALEAELAALEGRTFTIGEVK |
| | | GKLQGLIMKLHHLKDAC |
| 33 | F3-100n182 | CAAELAALEAELAALEGRPVRTVLTN |
| | | GKLMVLKSKLQELKHAC |
| 34 | F3-100n174 | CAAELAALEAELAALEGQKHFTLGGL |
| | | GKLIVLKKQLRILKCAC |
| 35 | F3-100n117 | CAAELAALEAELAALEGGAFLTVGRA |
| | | GKLMMLKQKLVRLKCAC |
| 36 | F3-100n458 | CAAELAALEAELAALEGENVYTLGGL |
| | | GKLMLLKYKLRELKGAC |
| 37 | F3-100n548 | CAAELAALEAELAALEGKDFRTIELV |
| | | GKLVLLKYKLNNLKEAC |
| 38 | F3-100n322 | CAAELAALEAELAALEGGRTPVVTTM |
| | | GKLMGLKLKLQQLKRAC |
| 39 | F3-100n386 | CAAELAALEAELAALEGETNMTVLMT |
| | | GKLLVLKHKLEHLKWAC |
| 40 | F3-100n50 | CAAELAALEAELAALEGASTPTLESR |
| | | GKLGSLIFKLRQLKVAC |
| 41 | F3-100n192 | CAAELAALEAELAALEGLHTLTLMQT |
| | | GKLLVLKMKLRELKWAC |
| 42 | F3-100n16 | CAAELAALEAELAALEGGAPVTIMGV |
| | | GKLMVLKEKLRNLKDAC |
| 43 | F3-100n3 | CAAELAALEAELAALEGSTARTLMQV |
| | | GKLEMLKYKLLELKCAC |
| 44 | F3-100n504 | CAAELAALEAELAALEGEVNMTRARA |
| | | GKLLVLKVKLHGLKQAC |
| 45 | F3-100n161 | CAAELAALEAELAALEVGRVKTLGRY |
| | | GKLMVLKHQLRNLKCAC |
| 46 | F3-100n250 | CAAELAALEAELAALEGLDYHTVLST |
| | | GKLMVLKRKLNILKLAC |

TABLE 3

| SEQ | | |
|---|---|---|
| No. | Clone | Sequence |
| 47 | FGFR_Y09 | CAAELAALEAELAALEGPPRPHLPPL |
| | | GKLFLLKFKLYLLKGAC |
| 48 | FGFR_Y13 | CAAELAALEAELAALEGPSRVGSRRR |
| | | GKLNLLKYKLHGLKLAC |

The present inventive peptide may be formed by the substitution, deletion and/or addition of one or several amino acid residues in the amino acid sequences represented by SEQ ID NOs: 1 to 3 and 4 to 48, as long as the effect of the present invention is not impaired. It is preferred that the number of amino acid residues to be substituted, deleted and/or added is no more than 10. More preferably, the number is 5 or less or 2 or less. For example, the amino acid residues indicated by X in SEQ ID NOs: 1 to 3 are suitable as such substitutions, deletions and/or additions.

The present inventive peptide can include peptides fused with a peptide that binds to heparan sulfate (HBD-100nX). The peptide that binds to heparan sulfate can include, for example, WQPPRARIG (SEQ ID NO: 49).

The fusion of the present inventive peptide with a peptide that binds to heparan sulfate can be performed directly or via a suitable linker. Such linkers can include, for example, polyamide linkers, polyethylene glycol (PEG) linkers, alkyl linkers, and polycarbonate linkers.

The present inventive peptide may be dimerized (100nX-Dimer). Such dimerization can be performed by two present inventive peptides directly or via a suitable linker. Such linkers can include, for example, polyamide linkers, polyethylene glycol (PEG) linkers, alkyl linkers, and polycarbonate linkers.

Use of HBD-100nX and 100nX-Dimer as the present inventive peptide can perform the induction of differentiation from pluripotent stem cells to somatic cells more efficiently.

The present inventive peptide may be molecularly modified with an appropriate compound or substance at its N- or C-terminus. Such molecular modification is typically performed for the purpose of fluorescent labeling, multimerization, etc. Compounds or the like for such molecular modification, for example, can include fluorescent compounds such as biotin, phage, FITC, etc.

The present inventive peptide including HBD-100nX, 100nX-Dimer, and peptides molecularly modified at N- or C-terminus can be easily produced artificially by chemical synthesis (or biosynthesis) according to conventional methods. As a conventional method, for example, either of the conventionally known solid-phase and liquid-phase synthesis methods may be employed. The solid-phase synthesis method in which Boc (t-butyloxycarbonyl) or Fmoc (9-fluorenylmethoxycarbonyl) is applied as a protecting group of the amino group is suitable. The present inventive peptide with a peptide chain having a desired amino acid sequence and/or modification (C-terminal amidation, etc.) moiety can be synthesized by a solid-phase synthesis method using a commercially available peptide synthesizer.

The present inventive peptide can also be biosynthesized based on genetic engineering methods. Specifically, DNA of the nucleotide sequence encoding the amino acid sequence of the desired present inventive peptide (containing ATG start codon) is synthesized. A recombinant vector having a gene construct for expression consisting of this DNA and various regulatory elements for the expression of the amino acid sequence (including a promoter, a ribosome binding site, a terminator, an enhancer, and various cis-elements that control the expression level) is then constructed according to the host cell.

The recombinant vector is introduced into a predetermined host cell by a general technique, and the host cell or a tissue or an individual containing the cell is cultured under predetermined conditions. In this way, the target polypeptide can be expressed and produced in the cell. The desired present inventive peptide can then be obtained by isolating and purifying the polypeptide from the host cell (or the culture medium, if secreted). By general techniques, the recombinant vector is introduced into a given host cell (e.g., yeast, insect cell), and the host cell or a tissue or an individual containing the cell is cultured under predetermined conditions. In this way, the target polypeptide can be expressed and produced in the cell. The desired peptide can then be obtained by isolating and purifying the polypeptide from the host cell (or the culture medium, if secreted).

For methods of constructing recombinant vectors and methods of introducing the constructed recombinant vectors into host cells, the conventional methods in the technical field can be employed as they are.

For example, a fusion protein expression system can be utilized for efficient mass production in host cells. That is, a gene (DNA) encoding the amino acid sequence of the desired present inventive peptide is chemically synthesized, and the synthetic gene is introduced into a suitable site of an appropriate fusion protein expression vector. The host cells (typically, E. coli) are then transformed with the vector. The resulting transformants are cultured to prepare the desired fusion protein. The protein is then extracted and purified. The purified fusion protein is then cleaved with a predetermined enzyme (protease), and the isolated target peptide fragment (the designed present inventive peptide) is recovered by affinity chromatography or the like. By using such a conventionally known fusion protein expression system, the present inventive peptide can be produced.

In addition, a template DNA (i.e., a synthetic gene fragment containing a nucleotide sequence encoding the amino acid sequence of a cell differentiation-inducing peptide) for a cell-free protein synthesis system is constructed, and the desired polypeptide can be synthesized in vitro by employing a so-called cell-free protein synthesis system with use of various compounds necessary for peptide synthesis (e.g., ATP, RNA polymerase, and amino acids and so on). As for the cell-free protein synthesis systems, for example, Shimizu et al. Nature Biotechnology, 19, 751-755 (2001), or Madin et al. Proc. Natl. Acad. Sci. USA, 97(2), 559-564 (2000) is instructive. On the basis of the technologies described in these papers, many companies are already performing contract production of polypeptides at the time of filing the present application, and kits for cell-free protein synthesis are commercially available.

Therefore, once the peptide chain is designed, the desired present inventive peptide can be easily synthesized and produced by a cell-free protein synthesis system according to its amino acid sequence.

The present inventive peptide may be in the form of a salt as long as the differentiation-inducing activity is not impaired. For example, acid- or base-added salts of the peptides obtained by an addition reaction with inorganic or organic acids, or inorganic or organic bases, which are usually used in accordance with a conventional method, can be used. Other salts (e.g., metal salts), hydrates, and solvates may also be used as long as they possess the differentiation-inducing activity. The present inventive peptide includes such salt forms.

### 2 Compositions According to the Present Invention

The present invention can include a composition for inducing differentiation of pluripotent stem cells into somatic cells, comprising the present inventive peptide (hereinafter referred to as the “present inventive composition”).

### 2.1 Pluripotent Stem Cells

Pluripotent stem cells are cells well known to persons skilled in the art and possess both the differentiation versatility to differentiate into all cell types of the body and the self-renewal ability to proliferate while maintaining their differentiation versatility.

Typical pluripotent stem cells can include induced pluripotent stem cells (iPS cells), embryonic stem cells (ES cells), and epiblast stem cells (EpiS), embryonic stem cells derived from cloned embryos obtained by nuclear transfer (ntES), sperm stem cells (GS cells), embryonic germ cells (EG cells), pluripotent cells derived from cultured fibroblasts or bone marrow stem cells (Muse cells), mesenchymal stem cells (MSCs).

### (1) iPS Cells

The iPS cells are pluripotent stem cells that are produced by introducing several types of reprogramming factors (typically three or four genes) into somatic cells. The iPS cells can be produced by introducing specific reprogramming factors into somatic cells in the form of DNA or proteins. Methods for producing the iPS cells are specifically described in, for example, WO2007/069666, WO2009/006930, WO2009/006997, WO2009/007852, WO2008/118820, Cell Stem Cell 3(5): 568-574 (2008), Cell Stem Cell 4(5): 381-384 (2009), Nature 454: 646-650 (2008), Cell 136(3): 411-419 (2009), Nature Biotechnology 26: 1269-1275 (2008), Cell Stem Cell 3: 475-479 (2008), Nature Cell Biology 11: 197-203 (2009), Cell 133(2): 250-264 (2008), Cell 131(5): 861-72 (2007), Science 318 (5858): 1917-20 (2007). They are artificial stem cells derived from somatic cells with characteristics similar to those of ES cells described below, such as pluripotency and the ability to proliferate through self-renewal.

The iPS cells can be selected based on the shape of the colonies formed. On the other hand, if a drug resistance gene that is expressed in conjunction with a gene that is expressed when somatic cells are initialized (e.g., Oct3/4, Nanog) is introduced as a marker gene, the iPS cells established can be selected by culturing in a culture medium containing the corresponding drug (selection culture medium). When the marker gene is a fluorescent protein gene, the iPS cells can be selected by observation under a fluorescence microscope. When the marker gene is a luminescent enzyme gene, the iPS cells can be selected by adding a luminous substrate. When the marker gene is a chromogenic enzyme gene, the iPS cells can be selected by adding a chromogenic substrate.

### (2) ES Cells

The ES cells are stem cells with pluripotency and the ability to proliferate by self-renewal, which are established from the internal cell mass of an early embryo (e.g., blastocyst) of mammals such as humans and mice. The ES cells are embryo-derived stem cells derived from the inner cell mass of a blastocyst, which is the embryo after the 8-cell stage of a fertilized egg, and have the ability to differentiate into all the cells that make up an adult body, so-called pluripotency, and the ability to proliferate through self-renewal. The ES cells can be established by removing the inner cell mass from the blastocyst of a fertilized egg of a target animal and culturing the inner cell mass on a fibroblast feeder. Maintenance of the cells in passaging culture can be performed by using a culture medium containing a substance such as leukemia inhibitory factor (Leukemia Inhibitory Factor (LIF)), basic fibroblast growth factor (bFGF), or the like.

The ES cells can include, for example, those of humans (Thomson J. A. et al., Science 282: 1145-1147 (1998), Biochem Biophys Res Commun. 345(3), 926-32 (2006)); those of primates such as rhesus monkeys and marmosets (Thomson J. A. et al., Proc. Natl. Acad. Sci. USA 92: 7844-7848 (1995), Thomson J. A. et al., Biol. Reprod. 55: 254-259 (1996)); those of rabbits (JP, 2000-508919, A)); those of hamsters (Doetshman T. et al., Dev. Biol. 127: 224-227 (1988)); those of pigs (Evans M. J. et al., Theriogenology 33: 125128 (1990), Piedrahita J. A. et al., Theriogenology 34: 879-891 (1990), Notarianni E. et al., J. Reprod. Fert. 40: 51-56 (1990), Talbot N. C. et al., Cell. Dev. Biol. 29A: 546-554 (1993)), those of sheep (Notarianni E. et al., J. Reprod. Fert. Suppl. 43: 255-260 (1991)); those of cattle (Evans M. J. et al., Theriogenology 33: 125-128 (1990), Saito S. et al., Roux. Arch. Dev. Biol. 201: 134-141 (1992)); those of minks (Sukoyan M. A. et al., Mol. Reorod. Dev. 33: 418-431 (1993)) and the like.

### (3) Other Pluripotent Stem Cells

The EpiS cells are pluripotent stem cells produced from the postimplantation epiblast.

The sperm stem cells are pluripotent stem cells derived from the testis and are the origin of spermatogenesis. Like the ES cells, these cells can be induced to differentiate into cells of various lineages and have characteristics, for example, that implantation into mouse blastocysts can produce chimeric mice. These cells can self-replicate in culture medium containing glial cell line-derived neurotrophic factor (GDNF), and can be obtained by repeated passaging under the same culture conditions as the ES cells.

The EG cells are established from primordial germ cells in the embryonic stage, and have pluripotency similar to that of the ES cells. They can be established by culturing primordial germ cells in the presence of substances such as LIF, bFGF, stem cell factor and the like. The EG cells can include, for example, human EG cells (Shamblott, et al., Proc. Natl. Acad. Sci USA 92: 7844-7848 (1995)).

The ntES cells are ES cells derived from cloned embryos produced by nuclear transfer technology and have almost the same characteristics as fertilized egg-derived ES cells.

The Muse cells are pluripotent stem cells produced by the method described in WO2011/007900.

Each of these pluripotent stem cells may be derived from humans or from animals such as mice, rats, cattle, pigs, and monkeys. They may also be naive or primed condition. They may be produced by culturing on mouse fibroblasts (e.g., mouse embryonic fibroblasts (MEFs)), or human neonatal or adult fibroblasts as feeder cells, or from feeder-less cultures.

### 2.2 Somatic Cells

The present inventive composition can be used to induce differentiation from pluripotent stem cells to somatic cells as described above.

The somatic cells to be induced to differentiate with the present inventive composition are not particularly limited and may be ectodermal, mesodermal, and endodermal cells. Specifically, ectoderm-derived cells can include corneal cells, neurons (dopamine-producing neurons, motor neurons, peripheral neurons, etc.), pigment epithelial cells, skin cells, and inner ear cells. Endoderm-derived cells can include hepatocytes, pancreatic progenitor cells, insulin-producing cells, cholangiocytes, alveolar epithelial cells, intestinal epithelial cells, etc. Mesoderm-derived cells can include cardiomyocytes, skeletal muscle cells, vascular endothelial cells, blood cells, bone cells, cartilage cells, renal progenitor cells, and renal epithelial cells. The somatic cells include not only finally differentiated mature cells but also cells in the process of differentiation that have not yet reached final differentiation. Among them, the present inventive composition is preferably used for inducing differentiation of neurons, cardiomyocytes, or hepatocytes.

In case the somatic cells are cardiomyocytes, such cardiomyocytes are defined as cells of the myocardium that have self-beating properties. The cardiomyocytes may also include cardiac progenitor cells, and may include those cells that are capable of giving rise to cardiomyocytes that form beating muscle and an electrically conducting tissue, and vascular smooth muscle. Said cardiomyocytes and cardiac progenitor cells may be mixed with each other or may be isolated cardiac progenitor cells.

The cardiomyocytes and cardiac progenitor cells can be confirmed to be induced by determining the expression levels of, for example, the myocardial marker cardiac troponin (cTNT or troponin T type 2), α-actinin 2 (ACTN2), αMHC (α myosin heavy chain), GATA4, CXCR4, Flk 1, and ANP. The cardiomyocytes may be a cell population that contains a higher proportion of cardiomyocytes relative to other cell types, and it is preferred that the proportion of cardiomyocytes in the cell population is no less than 30% or 50%.

### 2.3 Forms of the Present Inventive Composition

The present inventive composition need only to contain at least the present inventive peptide and can contain any other suitable ingredients.

Specifically, the present inventive composition can be, for example, a medium for cell culture (liquid or solid medium). For example, the present inventive composition can be made by adding the present inventive peptide to the general medium MEM (Minimum Essential Medium), DMEM (Dulbecco's Modified Eagle's Medium), DMEM/F12, or a modified medium thereof. Serum, proteins (albumin, transferrin, growth factors, etc.), amino acids, sugars, vitamins, fatty acids, antibiotics, and other substances effective in inducing differentiation of target somatic cells (e.g., liquid factors) may be added to the medium as the present inventive composition, if desired.

The present inventive composition may also be a kit for inducing (promoting) somatic cell differentiation containing the present inventive peptide. The kit may contain, in addition to the present inventive peptide, another liquid factor for inducing differentiation. The present inventive peptide and another liquid factor for inducing differentiation may be stored in separate containers or in the same container.

The present inventive composition can contain a variety of pharmaceutically acceptable carriers depending on the form of use, as long as the present inventive peptide can be retained in a state in which its differentiation-inducing activity is not lost. Carriers commonly used in peptide pharmaceuticals as diluents, excipients, or the like are preferred. Typical examples can include water, physiological buffers, and various organic solvents, although they may vary depending on the use and form of the present inventive composition. The carriers may be an aqueous alcohol (such as ethanol) solution of appropriate concentration, glycerol, or non-drying oil such as olive oil. They may also be liposomes. Alternatively, other ingredients that can be contained in the present inventive composition can include various fillers, bulking agents, binders, humectants, surface active agents, dyes, fragrances, antibiotics, and so on. Alternatively, other known cell differentiation inducing factors may be contained. Combination of the present inventive peptide and another cell differentiation inducing factor (retinoic acid, activin, bFGF, VEGF, BMP4, etc.) can promote (enhance) cell differentiation induction.

The form of the present inventive composition is not particularly limited. The form can include, for example, solution, suspension, semi-solid, solid, and gel forms. Typical forms can include, for example, liquid, suspension, emulsion, aerosol, foam, granule, powder, tablet, capsule, ointment, and water-based gel formulations. It can be lyophilized or granulated form for preparing a drug solution for use in injection, etc. by dissolving it in saline or an appropriate buffer solution (e.g., PBS) immediately before use.

Note that the process per se of preparing various forms of drugs (compositions) using the present inventive peptide (main ingredient) and various carriers (sub-ingredients) as materials should be in accordance with conventionally known methods.

### 3 Process for Producing Somatic Cells in Accordance with the Present Invention

The present invention also can include a process for producing somatic cells, comprising a step of applying a liquid factor containing a synthetic peptide having binding properties to the FGF receptor (hereinafter referred to as “FGFR binding peptide”) to the embryoid body of a pluripotent stem cell (hereinafter referred to as the “present inventive process”).

The above FGFR binding peptide is not particularly limited, as long as they would be determined by persons skilled in the art to have binding properties to the FGF receptor based on appropriate experimental results. Among the FGF receptors, peptides that bind to FGFR1 and/or FGFR3 are preferred. Binding to the FGF receptor can be confirmed, for example, by Surface Plasmon Resonance (SPR) or ELISA (Enzyme-Linked ImmunoSorbent Assay).

The FGFR binding peptide consisting of no more than 50 amino acid residues is appropriate. Those consisting of 30 to 50 or 40 to 50 amino acid residues are preferred. Those having the amino acid sequence of SEQ ID NO: 50 below in the molecular structure thereof are more preferred.

[Chem 7]
(SEQ ID No. 50)
C A A E L A A L E A E L A A L E G
•••

Specifically, the FGFR binding peptide can include, for example, the present inventive peptide, P3, C19, and Dekafin 1.

The FGFR binding peptide can be obtained in a manner of the following, for example. A phage surface-displayed peptide library is prepared by using appropriate phagemid vectors (e.g., pComb3, pCANTAB5E, and pSEX). The resulting phage surface-displayed library is then mixed with human Fc fragments for negative selection against Fc. This operation allows phage that bind to Fc in the phage library to be excluded. The phage library is then subjected to FGFR1 (FGF receptor 1)-Fc and the bound phage is recovered. Phagemid DNA is extracted from the recovered phage, and comprehensive analysis of the phagemid DNA is performed. Peptides are synthesized from the amino acid sequence of the obtained clones by Fmoc solid-phase synthesis or the like. Binding activity of the synthesized peptides is measured using the above-mentioned surface plasmon resonance method or ELISA, and the target FGFR binding peptide can be obtained based on the measurement results.

Liquid factors other than the FGFR binding peptide that can be used in the present inventive process depend on the target undifferentiated cell type and purpose (kind of differentiated cells to be produced), and can include, for example, bone morphogenetic protein 2 (BMP2), bone morphogenetic protein 4 (BMP7), bone morphogenetic protein 7 (BMP7), basic fibroblast growth factor (bFGF, also called fibroblast growth factor 2 (FGF2)), fibroblast growth factor 4 (FGF4), fibroblast growth factor 8 (FGF8), fibroblast growth factor 10 (FGF10), hepatocyte growth factor (HGF), platelet-derived growth factor BB (PDGF-BB), Wnt3a protein (Wnt3a), sonic hedgehog (Shh), vascular endothelial growth factor (VEGF), transforming growth factor-β (TGF-(β), Activin A, Oncostatin M (OSM), keratinocyte growth factor (KGF, also called fibroblast growth factor (FGF7)), glial-cell derived neurotrophic factor (GDNF), IWP-3 (CAS No. 687561-60-0), transferrin, and epidermal growth factor (EGF), etc. These liquid factors may be added to the culture medium or secreted by the cells in culture. One, two or more kinds of liquid factors may be used.

In the case of inducing differentiation in vivo of undifferentiated cells (e.g., iPS cells, ES cells, or other stem cells transplanted to a predetermined site), an appropriate amount of the present inventive composition (the present inventive peptide) can be supplied to the affected area (i.e., in vivo) in the desired amount, for example, as a liquid preparation. Alternatively, a solid form, such as a tablet, or a gel or aqueous jelly form, such as an ointment, can be administered to the affected area (i.e., a body surface such as a burn or wound). This can improve (promote) the efficiency of inducing differentiation of the target undifferentiated cells (e.g., stem cells or the like) to be differentiated that are typically present in or around the affected area in vivo. Note that the amount and frequency of addition are not particularly limited, as they can vary depending on the type of cell to be induced to differentiate, the site of existence, and other conditions.

By administering the present inventive composition (the present inventive peptide) to the necessary site in the living body, the ability of nerve regeneration, angiogenesis, skin regeneration, organ regeneration, and so on can be improved due to the differentiation-inducing activity thereof. In addition, promotion of differentiation into target cell types and organa (organs) enable, for example, improvement of skin tissue, early engraftment of transplanted organs, and early repair of wounds and burns caused by traffic accidents or other physical damage. It can also be used as a drug composition that contributes to the treatment of neurological diseases such as Parkinson's disease, cerebral infarction, Alzheimer's disease, paralysis of the body due to spinal cord injury, cerebral contusion, amyotrophic lateral sclerosis, Huntington's disease, brain tumors, retinal degeneration, for example, through regenerative medical approaches.

Furthermore, target differentiated cells (and differentiated tissues and organs) can be efficiently produced from cultured cell lines of material stem cells (iPS cells and ES cells). That is, by introducing the target differentiated cells (or tissues or organs comprising the differentiated cells) efficiently produced ex vivo (in vitro) by employing the production method disclosed herein (in vitro production method of differentiated cells or tissue bodies comprising the differentiated cells) into the affected area (i.e., into the patient's body) where repair or regeneration is required, it is possible to efficiently carry out the repair or regeneration.

The present inventive process comprises a step of applying a liquid factor containing the FGFR binding peptide to the embryoid body of said pluripotent stem cells, and can be performed in a conventional method in accordance with a differentiation induction protocol according to the type of somatic cells desired, and the like. The differentiation induction is typically performed by culturing the cells. The culture method is selected from, for example, an adhesion culture method, a floating culture method, or a suspension culture method, according to the desired cells (e.g., cardiomyocytes) to be induced to differentiate from the pluripotent stem cells.

Somatic cells that can be produced by the present inventive process can include, for example, the somatic cells mentioned above. Among them, when mesoderm cells, especially cardiomyocytes, are produced by the present inventive process, it is preferable to use the present inventive peptide as an FGFR binding peptide.

For example, when the somatic cells obtained by the present inventive process are cardiomyocytes, said cardiomyocytes can be used as a therapeutic agent for the treatment of cardiac diseases in animals (preferably humans). The treatment of cardiac disease may be performed, for example, by suspending the obtained cardiomyocytes in saline or the like and administering them directly into the myocardial layer of the patient's heart, or by forming the obtained cardiomyocytes into sheets and attaching them to the patient's heart. In the former case, the cells may be administered alone or preferably with scaffold materials that promote viability. Here, the term “scaffold material” is exemplified by, but not limited to, biologically derived components such as collagen or synthetic polymers such as polylactic acid as an alternative. Administration of the myocardial sheet is accomplished by placing it to cover the desired portion. The placement of the sheet to cover the desired portion can be performed using techniques known in the art. Upon placement, if the desired portion is large, it may be placed around the tissue. The administration may be performed several times over the same area to achieve the desired effect. In the case of multiple placements, it is desirable to give sufficient time for the desired cells to engraft into the tissue and for angiogenesis to occur.

The mechanism of treatment of cardiac disease may be an effect produced by the myocardial sheet engraftment, or it may be an indirect effect that is not caused by cell engraftment (e.g., the effect of mobilization of recipient-derived cells to the site of injury trough secretion of an attractant). In the treatment of cardiac disease, the cardiac sheet may contain cell scaffold materials (scaffold) such as collagen, fibronectin, laminin, etc., in addition to cardiomyocytes. Alternatively, it can also contain any cell type(s) in addition to cardiomyocytes. Cardiac diseases that may be treated in the present invention include, but are not limited to, defects due to diseases or disorders such as heart failure, ischemic heart disease, myocardial infarction, myocardiopathy, myocarditis, hypertrophic cardiomyopathy, dilated phase hypertrophic cardiomyopathy, and dilated cardiomyopathy.

In the present invention, the number of cardiomyocytes used in the treatment of cardiac disease is not limited as long as the cardiomyocytes or cardiomyocyte sheets to be administered are present in such an amount that they are effective in the treatment of cardiac disease, and may be increased or decreased to suit the size of the affected area or the trunk.

### 4 Method of Inducing Differentiation of Pluripotent Stem Cells into Somatic Cells in Accordance with the Present Invention

The present invention can also include a method for inducing differentiation of pluripotent stem cells into somatic cells, comprising a step of applying a liquid factor containing an FGFR binding peptide to the embryoid body of pluripotent stem cells (hereinafter referred to as the “present inventive differentiation induction method”).

The above FGFR binding peptide can be the same as those mentioned above. The concepts of terms such as liquid factors other than the FGFR binding peptide, pluripotent stem cells, and somatic cells are the same as those described above.

The present inventive differentiation induction method can be carried out in a conventional method, except for the use of liquid factors containing FGFR binding peptides. That is, the present inventive differentiation induction method can be carried out by applying liquid factors including FGFR binding peptides to the embryoid body of pluripotent stem cells at the appropriate time according to the differentiation induction protocol according to the desired somatic cell type, etc.

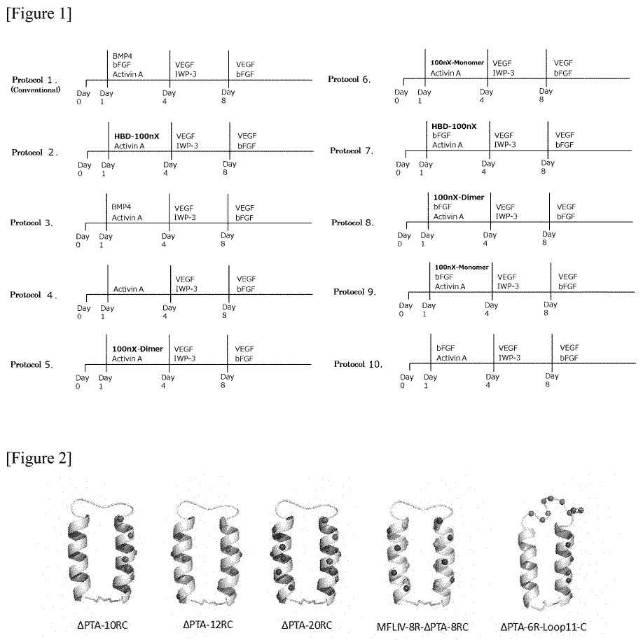

Specifically, for example, in the case of inducing differentiation of pluripotent stem cells into cardiomyocytes, the differentiation induction protocol shown in FIG. 1 (Protocols 2, and 5 to 9) can be followed. In FIG. 1, Protocol 1 represents the conventional protocol for inducing differentiation of cardiomyocytes in the conventional technology of Patent Document 1 (conventional protocol).

As shown in FIG. 1, in the conventional protocol, one day after the start of induction, liquid factors such as activin A, BMP4, and bFGF are applied to pluripotent stem cells, whereas in the present inventive differentiation induction method, BMP4 and bFGF are not necessarily required. With only the present inventive peptide and activin A, differentiation induction into cardiomyocytes can be performed. Therefore, the present inventive differentiation induction method can efficiently induce differentiation into somatic cells using relatively inexpensive medium-molecular weight peptides without using expensive proteins such as BMP4 and bFGF.

The present inventive differentiation induction method can also be performed with the addition of other liquid factors, such as bFGF, one day after the start of induction, as in conventional protocols. By doing that, in general, induction into somatic cells can be performed more efficiently.

More specifically, the present inventive differentiation induction (especially, the method of inducing differentiation into cardiomyocytes) may comprise, for example, the following steps.

### (1) The Process of Forming Embryoid Bodies from Pluripotent Stem Cells

It is preferable to dissociate the pluripotent stem cells that have formed a colony into single cells and then form embryoid bodies. In the process of dissociating pluripotent stem cells, cells that adhere to each other and form a population are dissociated (separated) into individual cells. Methods for dissociating pluripotent stem cells can include, for example, mechanical dissociation methods, and dissociation methods using dissociation solutions with protease and collagenase activities (e.g., Accutase™ and Accumax™) or dissociation solutions with only collagenase activity. Preferably, dissociation solutions with protease and collagenase activity (especially, preferably Accumax) are used to dissociate pluripotent stem cells.

Methods of forming embryoid bodies can include, for example, floating culture of dissociated pluripotent stem cells in culture dishes whose surfaces have not been artificially treated for the purpose of improving cell adhesion (e.g., coating treatment with extracellular matrix such as Matrigel (trade name), collagen, gelatin, laminin, heparan sulfate proteoglycan, or entactin), or floating culture with dishes artificially treated to inhibit adhesion (e.g., coating treatment with polyhydroxyethyl methacrylate (poly-HEMA)).

The number of pluripotent stem cells suitably used to form embryoid bodies for the purpose of inducing cardiomyocytes is, for example, from 1,000 to 16,000, preferably from 2,000 to 8,000 cells.

### (2) Process of Culturing Embryoid Bodies in Culture Medium

The culture medium used in this process can be prepared by adding the FGFR binding peptide such as the present inventive peptide, Activin A, etc. to the basic medium used for culturing animal cells.

Basic medium includes, for example, IMDM medium, Medium 199 medium, Eagle's Minimum Essential Medium (EMEM) medium, αMEM medium, Dulbecco's modified Eagle's Medium (DMEM) medium, Ham's F12 medium, RPMI 1640 medium, Fischer's medium, Neurobasal Medium (Life Technologies), StemPro34 (Invitrogen), and mixtures of these media, etc. The medium may contain serum or be serum-free. If necessary, the medium may contain one or more serum substitute(s), for example, albumin, transferrin, Knockout Serum Replacement (KSR) (serum substitute for FBS during ES cell culture), N2 supplement (Invitrogen), B27 supplement (Invitrogen), fatty acids, insulin, collagen precursors, trace elements, 2-mercaptoethanol, and 1-thiol glycerol; and may also contain one or more substance(s) such as lipids, amino acids, L-glutamine, Glutamax (Invitrogen), nonessential amino acids, vitamins, growth factors, low molecular weight compounds, antibiotics, antioxidants, pyruvate, buffers, and inorganic salts. Preferred basic medium is StemPro34 containing transferrin, 1-thiol glycerol, L-glutamine, and ascorbic acid.

The concentration of the FGFR binding peptide such as the present inventive peptide in the process varies depending on the type of the present inventive peptide and the FGFR binding peptide, and is appropriately in the range of 1 pM to 100 μM, preferably in the range of 50 pM to 100 nM, and more preferably in the range of 50 pM to 5 nM.

In the case that Activin A is used in this process, the concentration of Activin A is appropriately in the range of 1 ng/mL to 100 ng/mL, preferably in the range of 1 ng/mL to 50 ng/mL, and more preferably in the range of 10 ng/mL to 20 ng/mL.

In the case that bFGF is used in this process, the concentration of bFGF is appropriately in the range of 1 ng/mL to 100 ng/mL, and more preferably in the range of 1 ng/mL to 20 ng/mL.

In the case that BMP4 is used in this process, the concentration of BMP4 is appropriately in the range of 1 ng/mL to 100 ng/mL, preferably in the range of 1 ng/mL to 50 ng/mL, and more preferably in the range of 1 ng/mL to 20 ng/mL.

The culture conditions are as follows.

An incubation temperature of about 30 to 40° C. is appropriate, preferably about 37° C., and culturing under hypoxic conditions is desirable. Here, hypoxic conditions are those with an oxygen partial pressure lower than the oxygen partial pressure in the atmosphere (20%), for example, between 1% and 15%. Preferably it is 5%. Cultivation is carried out in an atmosphere of air containing CO₂, and the concentration of CO₂ is preferably from about 2 to 5%.

The culture period may be, for example, from 1 to 7 days, and considering the efficiency of establishment of cardiomyocytes, from 1 to 5 days, from 1.5 to 5 days, or 2 to 4 days are preferred.

### (3) Process of Forming Embryoid Bodies by Reaggregation through Culture in Culture Medium Containing VEGF and Wnt Inhibitor

The number of cells used to form embryoid bodies by reaggregation is not limited, as long as the cells can adhere to each other and form a cell mass. No less than 1,000 cells but no more than 20,000 cells are appropriate, and 10,000 cells are preferred. As in step (1), the cells are preferably subjected to floating culture in culture vessels whose surfaces have not been artificially treated to improve cell adhesion, or in culture vessels that have been artificially treated to inhibit adhesion.

The culture medium used in this process can be prepared by adding VEGF and Wnt inhibitors to the basic medium used for culturing animal cells. The basic medium can include, for example, IMDM medium, Medium 199 medium, Eagle's Minimum Essential Medium (EMEM) medium, αMEM medium, Dulbecco's modified Eagle's Medium (DMEM) medium, Ham's F12 medium, RPMI 1640 medium, Fischer's medium, Neurobasal Medium (Life Technologies), StemPro34 (Invitrogen), and mixtures of these media, etc. The medium may contain serum or be serum-free. If necessary, the medium may contain one or more serum substitute(s), for example, albumin, transferrin, Knockout Serum Replacement (KSR) (serum substitute for FBS during ES cell culture), N2 supplement (Invitrogen), B27 supplement (Invitrogen), fatty acids, insulin, collagen precursors, trace elements, 2-mercaptoethanol, 1-thiol glycerol; and may also contain one or more substance(s) such as lipids, amino acids, L-glutamine, Glutamax (Invitrogen), nonessential amino acids, vitamins, growth factors, low molecular weight compounds, antibiotics, antioxidants, pyruvate, buffers, inorganic salts. Preferred basic medium is StemPro34 containing transferrin, 1-thiol glycerol, L-glutamine, and ascorbic acid.

The term “Wnt inhibitor” refers to a substance that inhibits the signaling pathway that leads from Wnt binding to its receptor to the accumulation of β-catenin. It is not particularly limited, as long as it is a substance that inhibits binding to the Frizzled family of receptors, or a substance that promotes the degradation of β-catenin, and can include, for example, the DKK1 protein (e.g., in humans, NCBI accession number: NM_012242), sclerostin (e.g., in humans, NCBI accession number: NM_025237), IWR-1 (Merck Millipore), IWP-2 (Sigma-Aldrich), IWP-3 (Sigma-Aldrich), IWP-4 (Sigma-Aldrich), PNU-74654 (Sigma-Aldrich), XAV939 (Sigma-Aldrich), and their derivatives. Of these, IWP-3 or IWP-4 is preferred.

The concentration of Wnt inhibitors such as IWP-3 or IWP-4 in the culture medium is not particularly limited, as long as the concentration is capable of inhibiting Wnt, and is appropriately in the range of 1 nM to 50 μM, preferably in the range of 10 nM to 25 μM, and more preferably in the range of 100 nM to 10 μM.

The concentration of VEGF used in this process is, for example, appropriately in the range of 1 ng/mL to 100 ng/mL, preferably in the range of 1 ng/mL to 50 ng/mL, and more preferably in the range of 1 ng/mL to 20 ng/mL.

In this process, BMP inhibitors and/or TGFβ inhibitors may also be added to the basic medium.

The term “BMP inhibitor” can include protein-based inhibitors such as Chordin, Noggin and Follistatin, Dorsomorphin (i.e., 6-[4-(2-piperidin-1-yl-ethoxy) phenyl]-3-pyridin-4-yl-pyrazolo[1,5-a] pyrimidine), and its derivatives; and LDN-193189 (i.e., 4-(6-(4-(piperazin-1-yl) phenyl) pyrazolo[1,5-a] pyrimidin-3-yl) quinoline). Dorsomorphin and LDN-193189 are commercially available, and are manufactured by Sigma-Aldrich and Stemgent, respectively. Dorsomorphin may be preferable.

The concentration of BMP inhibitors such as Dorsomorphin in the culture medium is not particularly limited, as long as the concentration is capable of inhibiting BMP, and is appropriately in the range of 1 nM to 50 nM.

The term “TGFβ inhibitor” refers to a substance that inhibits the signaling pathway that leads from TGFβ binding to its receptor to SMAD. It is not particularly limited, as long as it is a substance that inhibits binding to the ALK family of receptors, or a substance that inhibits the phosphorylation of SMAD by the ALK family, and can include, for example, Lefty-1 (NCBI accession numbers: NM_010094 for mouse, NM_020997 for human, are exemplified), SB431542, SB202190 (R. K. Lindemann et al., Mol. Cancer, 2003, 2:20), SB505124 (GlaxoSmithKline), NPC30345, SD093, SD908, SD208 (Scios), LY2109761, LY364947, LY580276 (Lilly Research Laboratories), A-83-01 (WO 2009146408) and their derivatives. SB431542 is preferable.

The concentration of TGF13 inhibitor such as SB431542 in the culture medium is not particularly limited, as long as the concentration is capable of inhibiting ALK5, and is appropriately in the range of 1 nM to 50 nM.

The culture conditions are as follows.

An incubation temperature is appropriately about 30 to 40° C., preferably about 37° C., and culturing under hypoxic conditions is desirable. Here, hypoxic conditions are those with an oxygen partial pressure lower than the oxygen partial pressure in the atmosphere (20%), for example, between 1% and 15%. Preferably it is 5%. Cultivation is carried out in an atmosphere of air containing CO₂, and the concentration of CO₂ is preferably from about 2 to 5%.

The culture period is preferably be 4 days or longer, though there is no upper limit because the establishment of cardiomyocytes is not affected by prolonged culture. This allows the embryoid bodies formed by reaggregation to differentiate into cardiomyocytes.

### (4) Process of Culturing in a Culture Medium Containing VEGF and bFGF

The culture medium used in this process can be prepared by adding VEGF and bFGF to the basic medium used for culturing animal cells. The basic medium can include, for example, IMDM medium, Medium 199 medium, Eagle's Minimum Essential Medium (EMEM) medium, αMEM medium, Dulbecco's modified Eagle's Medium (DMEM) medium, Ham's F12 medium, RPMI 1640 medium, Fischer's medium, Neurobasal Medium (Life Technologies), StemPro34 (Invitrogen), and mixtures of these media, etc. The medium may contain serum or be serum-free. If necessary, the medium may contain one or more serum substitute(s), for example, albumin, transferrin, and Knockout Serum Replacement (KSR) (serum substitute for FBS during ES cell culture), N2 supplement (Invitrogen), B27 supplement (Invitrogen), fatty acids, insulin, collagen precursors, trace elements, 2-mercaptoethanol, 1-thiol glycerol; and may also contain one or more substance(s) such as lipids, amino acids, L-glutamine, Glutamax (Invitrogen), nonessential amino acids, vitamins, growth factors, low molecular weight compounds, antibiotics, antioxidants, pyruvate, buffers, inorganic salts. Preferred basic medium is StemPro34 containing transferrin, 1-thiol glycerol, L-glutamine, and ascorbic acid.

The concentration of VEGF used in this process is, for example, appropriately in the range of 1 ng/mL to 100 ng/mL, preferably in the range of 1 ng/mL to 50 ng/mL, and more preferably in the range of 1 ng/mL to 10 ng/mL.

The concentration of bFGF used in this process is, for example, appropriately in the range of 1 ng/mL to 100 ng/mL, preferably in the range of 1 ng/mL to 50 ng/mL, and more preferably in the range of 1 ng/mL to 10 ng/mL.

The culture conditions are as follows.

An incubation temperature is appropriately about 30 to 40° C., preferably about 37° C., and culturing under hypoxic conditions is desirable. Here, hypoxic conditions are those with an oxygen partial pressure lower than the oxygen partial pressure in the atmosphere (20%), for example, between 1% and 15%. Preferably it is 5%. Cultivation is carried out in an atmosphere of air containing CO₂, and the concentration of CO₂ is preferably from about 2 to 5%.

The culture period is preferably be 12 days or longer, though there is no upper limit because the establishment of cardiomyocytes is not affected by prolonged culture. Further culturing the cells obtained in step (3) according to step (4) improves the efficiency of differentiation into cardiomyocytes.

### EXAMPLE

Hereinafter, the present invention will be specifically described with reference to Examples, but the present invention is not limited by the Examples in any way.

### [Example 1] Synthesis of the Present Inventive Peptide

### (1) Preparation of Phage Surface-Displayed Peptide Libraries

First, using the phagemid vector pComb3 (see Barbas, C. et al., Assembly of Combinatorial Antibody Libraries on Phage Surfaces: The Gene III Site., Proc. National Acad. Sci., 88, 7978-7982 (1991), and Fujii, I. et al., Evolving Catalytic Antibodies in a Phage-Displayed Combinatorial Library., Nat. Biotechnol. 16, 463-467 (1998)), seven phage surface-displayed peptide libraries (ΔPTA-1 ORC, ΔPTA-12RC-1, ΔPTA-12RC-2, ΔPTA-20RC, ΔPTA-6R-Loop11-C, MFLIV-8R-ΔPTA-8RC-1, MFLIV-8R-ΔPTA-8RC-2) were prepared (FIG. 2). The peptide chains constituting the library have the amino acid sequences shown in Table 4 below, respectively.

TABLE 4

| Library | | Degenerate |
|---|---|---|
| Name | Library Seqence | codon |
| Δ PTA- | CAAELAALEAELAALEGGGGGGG---- | X:NDK |
| 10RC | XLXXLXXXLXXLXXAC | |
| Δ PTA- | CAXELXALXXELXALXGGGGGGG---- | X:NDK |
| 12RC-1 | KLXALXXKLXALXXAC | |
| Δ PTA- | CAXXLAXLEXXLAXLEGGGGGGG---- | X:NDK |
| 12RC-2 | KLXALXXKLXALXXAC | |
| Δ PTA- | CAXXLXXLXXXLXXLXGGGGGGG---- | X:NDK |
| 20RC | XLXXLXXXLXXLXXAC | |
| Δ PTA- | CAAELAALEAELAALEGXXXXXXXXXG | X:NNK, |
| 6R- | KLZZLKZKLZZLKZAC | Z:NDK |
| Loop11- | | |
| C | | |
| MFLIV- | CAAXZAXZEAXZAXZEGGGGGGG---- | X:NDK, |
| 8R-Δ | XZAXZKAXZAXZKAAC | Z:DTS |
| PTA- | | |
| 8RC-1 | | |
| MFLIV- | CAAEZXAZXAEZXAZXGGGGGGG---- | X:NDK, |
| 8R-Δ | KZXAZXAKZXAZXAAC | Z:DTS |
| PTA- | | |
| 8RC-2 | | |

These peptide chains were constructed from the amino acid sequence of peptide YT-1 consisting of the known sequence (AELAALEAELAALEGGGGGGGKLAALKAKLAALKA; SEQ ID NO: 51). The amino acid X in the table represents an amino acid that is not considered to be involved in the maintenance of steric structure in the two α-helices, each consisting of 14 amino acid residues constituting the HLH (helix-loop-helix) structure, and can be substituted by an arbitrary amino acid. In addition, each peptide chain constituting the library has two peptide CA added to the N-terminus of α-helix at the N-terminal side and two peptide CA added to the C-terminus of α-helix at the C-terminal side. Note that in Table 4, ΔPTA-6R-Loop11-C is a randomized amino acid sequence in which the amino acid sequence of the loop portion of YT-1 is elongated.

### (2) Biopanning

The phage surface-displayed libraries obtained above were mixed, and biopanning against FGFR1 (FGF receptor 1)-Fc chimeras was performed. First, as a negative selection, 10¹² cfu of peptide-presenting phages in 200 μl of PBS (pH 7.4) were acted on a plate immobilized with hIgG Fc and phages that did not bind to hIgG Fc were collected. The collected phage library was mixed with 100 nM of FGFR3 solution and allowed to react overnight at 4° C. (Table 5: Panning Round 1).

TABLE 5

| Pannning | Target | | Incubation | Washing | | Output |
|---|---|---|---|---|---|---|
| Round | conc. | Negative-Selection | time | condition | Elution | phage |
| 1 | 100 nM | 10 μg/mL hIgG Fc | Overnight | PBST 3 | Gly-HCl, pH 2.0 | 4.6 × 10⁸ |
| | | 2 hr at R.T. | at 4° C. | times | 10 min at R.T. | |
| 2 | 100 nM | 10 μg/mL hIgG Fc | Overnight | PBST 5 | Gly-HCl, pH 2.0 | 1.3 × 10⁷ |
| | | 2 hr at R.T. | at 4° C. | times | 10 min at R.T. | |
| 3 | 100 nM | 10 μg/mL hIgG Fc | Overnight | PBST 10 | Gly-HCl, pH 2.0 | 1.0 × 10⁵ |
| | | 2 hr at R.T. | at 4° C. | times | 10 min at R.T. | |
| 4 | 100 nM | 10 μg/mL hIgG Fc | Overnight | PBST 15 | Gly-HCl, pH 2.0 | 2.7 × 10⁸ |
| | | 2 hr at R.T. | at 4° C. | times | 10 min at R.T. | |
| 5 | 100 nM | 10 μg/mL hIgG Fc | Overnight | PBST 20 | Gly-HCl, pH 2.0 | 1.5 × 10⁸ |
| | | 2 hr at R.T. | at 4° C. | times | 10 min at R.T. | |

After the reaction, the binding phages were captured by magnetic beads modified with protein A, and washed three times with PBS-T, followed by eluting the bound phages with Gly-HCl (pH 2.0). After neutralizing with 2M Tris, the phages were infected to E. coli. After 4 hours of incubation, helper phages were added, and the E. coli were allowed to produce phages overnight. Next, phage solution was prepared from the culture supernatant. The prepared phage solution was mixed with FGFR3 solution and allowed to react overnight at 4° C. (Table 5: Panning Round 2). After the reaction, the bound phages were eluted. Phage solution prepared in the same way was mixed with FGFR3 solution and allowed to react at 4° C. for 1 hour (Table 5: Panning Round 3). After the reaction, the bound phages were eluted. This operation was repeated two more times, and after Panning Round 5, the bound phages were eluted. Note that as the rounds proceeded, the number of washes with PBS-T was increased. (Table 5). The titer of the Output phage in each round was also determined. The titer of phage was determined from the number of colonies obtained by incubating the E. coli TG1 strain contacted with the phage at 37° C. overnight. The results are shown on the right side of Table 5.

### (3) Determination of Amino Acid Sequence

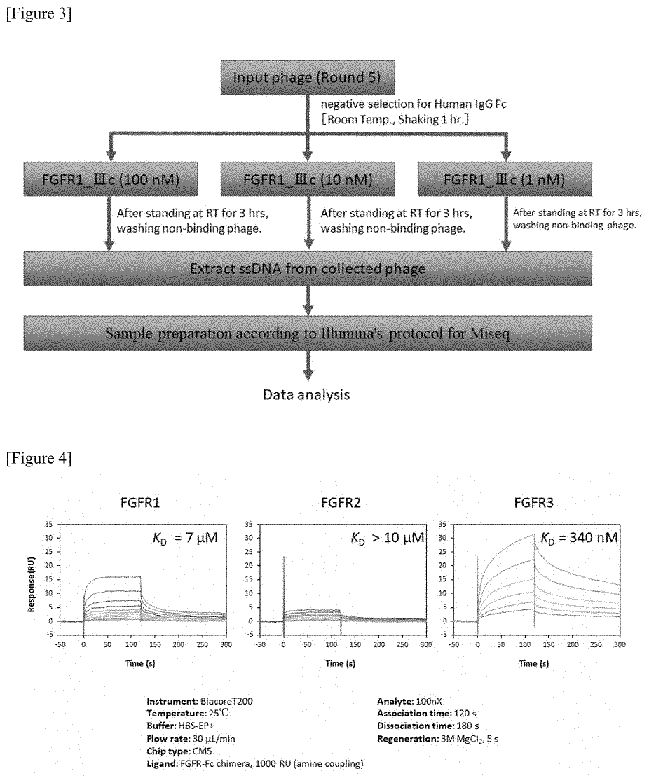

As shown in FIG. 3<Input phage (Round 5)>, the eluted phage library after panning was subjected to Human IgG Fc-immobilized plate, and negative selection was performed by collecting unbound phages. The phage library was then acted to FGFR1 prepared to concentrations of 100 nM, 10 nM, and 1 nM. Bound phages were then collected. The phagemid DNA of the collected phages was extracted, samples were prepared according to Illumina's protocol for Miseq, and a comprehensive analysis of the phagemid DNA was performed. Among the sequences obtained, 23 sequences with high incidence rates were selected (Table 6).

TABLE 6

| | | | 1 nM | | 10 nM | | 100 nM | |
|---|---|---|---|---|---|---|---|---|
| SEQ | | | Num. of | 1 nM | Num. of | 10 nM | Num. of | 100 nM |
| No. | ID | Library | reads | Ranking | reads | Ranking | reads | Ranking |
| 4 | 100n17 | Uncorrected | 8443 | 1 | 10095 | 1 | 8500 | 1 |
| 5 | 100n85 | dPTA-6R-Loop11C | 6769 | 2 | 9230 | 2 | 7482 | 2 |
| 6 | 100n82 | dPTA-6R-Loop11C | 6561 | 3 | 7109 | 4 | 4896 | 5 |
| 7 | 100n83 | dPTA-6R-Loop11C | 4939 | 5 | 6581 | 5 | 3079 | 6 |
| 8 | 100n8 | dPTA-6R-Loop11C | 4243 | 7 | 3717 | 7 | 1974 | 8 |
| 9 | 100n9 | dPTA-6R-Loop11C | 6403 | 4 | 7169 | 3 | 6135 | 3 |
| 10 | 100n368 | dPTA-6R-Loop11C | 2077 | 8 | 2177 | 9 | 1695 | 11 |
| 11 | 100n781 | dPTA-6R-Loop11C | 352 | 26 | 262 | 48 | 228 | 40 |
| 12 | 100n423 | dPTA-6R-Loop11C | 561 | 21 | 867 | 19 | 439 | 26 |
| 13 | 100n12 | dPTA-6R-Loop11C | 273 | 30 | 267 | 46 | 258 | 34 |
| 14 | 100n606 | dPTA-6R-Loop11C | 872 | 14 | 894 | 18 | 609 | 18 |
| 15 | 100n1793 | dPTA-6R-Loop11C | 493 | 23 | 493 | 29 | 395 | 29 |
| 16 | 100n417 | Uncorrected | 721 | 18 | 1010 | 15 | 762 | 14 |
| 17 | 100n1074 | dPTA-6R-Loop11C | 756 | 16 | 688 | 23 | 333 | 31 |
| 18 | 100n108 | dPTA-6R-Loop11C | 873 | 13 | 1155 | 14 | 464 | 22 |
| 19 | 100n182 | Uncorrected | 1428 | 10 | 1468 | 12 | 937 | 13 |
| 20 | 100n84 | dPTA-6R-Loop11C | 795 | 15 | 854 | 20 | 563 | 19 |
| 21 | 100n51 | dPTA-6R-Loop11C | 704 | 19 | 1000 | 16 | 649 | 16 |
| 22 | 100n36 | dPTA-6R-Loop11C | 483 | 24 | 677 | 24 | 398 | 28 |
| 23 | 100n1314 | dPTA-6R-Loop11C | 260 | 34 | 406 | 31 | 238 | 38 |
| 24 | 100n1543 | Uncorrected | 233 | 37 | 276 | 43 | 216 | 43 |
| 25 | 100n1348 | dPTA-6R-Loop11C | 349 | 27 | 473 | 30 | 346 | 30 |
| 26 | 100n222 | dPTA-6R-Loop11C | 525 | 22 | 776 | 22 | 541 | 20 |

### (4) Synthesis of Peptide and Confirmation of Binding to FGF Receptor

One clone was selected from the amino acid sequences of the clones obtained above, and the peptide of 100nX (X is 9: SEQ ID NO: 9, the same shall apply hereinafter.) was synthesized. The peptide was synthesized based on the Fmoc solid-phase synthesis method. To measure the binding activity of the peptides, according to the operation manual of the SPR instrument Biacore T200 (GE Healthcare), FGFR1 was immobilized as a ligand to the instrument's sensor chip CMS using the amine coupling method. In addition, to confirm binding to other FGFR species, FGFR2 or FGFR3-immobilized sensor chips were also prepared. Ethanolamine was immobilized as a reference, and the samples were measured as analyte.

All measurements were performed at 25° C. and HBS-EP+ was used as the running buffer. After injecting the running buffer dissolving the peptide into the CMS sensor chip immobilized with various FGFRs (flow rate 30 μl/min, 2 min), the peptide was dissociated by flowing the running buffer for 3 min. The obtained sensorgram was directly curve-fitted with the reaction equation, and the rate constant was calculated by a nonlinear least-squares method. Dissociation constants were calculated by kinetic analysis using Biacore T200 Evaluation Software (GE Healthcare). A 1:1 binding model was used in the analysis. The results are shown in FIG. 4.

As shown in FIG. 4, 100nX has a high binding affinity to the FGFR.

### [Example 2] Differentiation Induction of iPS Cells into Cardiomyocytes (Evaluation of HBD-100nX)

Next, the heparan sulfate binding peptide WQPPRARIG (SEQ ID NO: 49) fused to 100nX (HBD-100nX) was synthesized and applied to a system for inducing differentiation of iPS cells into cardiomyocytes. The used iPS cells were the “iPS cell-KAC, hFB(N)” (KAC, product number; IPS-F001) (hereinafter referred to as “iPS-hFB”). First of all, at the start of differentiation induction (Day 0), iPS-hFBs seeded into 100φ dishes on 7 days before the start of differentiation induction (Day-7) were washed with 4 ml of PBS. Three ml of 0.5xTrypLE Select was added, and allowed to stand for 3 minutes under 37° C. After confirming that the cells had become spherical, 0.5xTrypLE Select was removed. The cells were further washed with 4 ml of PBS and 4 ml of differentiation induction medium (STEMPRO34 (Invitrogen) supplemented with 1% L-Glutamine (Invitrogen), 150 μg/mL Transferrin (Roche), 50 μg/mL Ascorbic Acid (sigma), 3.9×10⁻³% MTG (1-Thyoglycerol) (sigma), 10 μM Rock inhibitor (Y-27632, Wako), 5% matrigel (Corning), and 2 ng/mL BMP4 (R&D)) was added. Cells were then detached with a cell scraper and isolated by pipetting. The cells were seeded into 96-well round-bottom plates at 8000 cells/well and incubated under 37° C., 5% CO₂ conditions.

The next day (Day 1), STEMPRO34 supplemented with 1% L-Glutamine, 150 μg/mL Transferrin, 50 μg/mL Ascorbic Acid, 3.9×10⁻³% MTG, 10 ng/mL BMP4, 5 ng/mL bFGF (R&D) and 6 ng/mL Activin A (R&D) was added at 100 μl/well (conventional protocol; FIG. 1, Protocol 1).

On the other hand, in the groups acted with peptide samples, STEMPRO34 supplemented with 1% L-Glutamine, 150 μg/mL Transferrin, 50 μg/mL Ascorbic Acid, 3.9×10⁻³% MTG, 0.05 μM or 0.5 μM HBD-100nX, and 6 ng/mL Activin A (R&D) was added at 100 μl/well (the present inventive differentiation induction method; FIG. 1, protocol 2). In addition, as control groups, the group in which STEMPRO 34 supplemented with 1% L-Glutamine, 150 μg/mL Transferrin, 50 μg/mL Ascorbic Acid, 3.9×10⁻³% MTG, 10 ng/mL BMP4, and 6 ng/mL Activin A (R&D) was added at 100 μl/wells (FIG. 1, protocol 3), and the group in which STEMPRO 34 supplemented with 1% L-Glutamine, 150 μg/mL Transferrin, 50 μg/mL Ascorbic Acid, 3.9×10⁻³% MTG, and 6 ng/mL Activin A (R&D) was added at 100 μl/well (FIG. 1, protocol 4) were prepared. The cells were then incubated under 37° C. and 5% CO₂ conditions for 3 days.

Subsequently (Day 4), the resulting EBs were centrifuged at 200 g for 3 minutes to remove the medium. PBS was added and the supernatant was removed by centrifuging at 200 g for 3 minutes. After Accumax was added and allowed to stand at 37° C. for 5 minutes, single cells were dissociated by pipetting, 2 mL of STEMPRO34 (Day 4 medium for myocardial induction) supplemented with 1% L-Glutamine, 150 μg/mL Transferrin, 50 μg/mL Ascorbic Acid, 3.9×10⁻³% MTG, 10 ng/mL VEGF and 1 μM IWP-3 was added, and the supernatant was removed by centrifuging at 200 g for 3 minutes. The cells were suspended in Day 4 medium for myocardial induction, and after the number of cells was measured, the cells were seeded in 96-well round-bottom plates at 10000 cells/well. The cells were then incubated under 37° C. and 5% CO₂ conditions for 4 days.

Subsequently (Day 8), the cell clusters were transferred in batches from 96-well plates to 24-well plates (no more than 8 cell clusters per well). After they were allowed to spontaneously sediment, the medium was removed. Then, STEMPRO34 (Day 8 medium for myocardial induction) supplemented with 1% L-Glutamine, 150 μg/mL Transferrin, 50 μg/mL Ascorbic Acid, 3.9×10⁻³% MTG, 10 ng/mL VEGF and 5 ng/mL bFGF was added, and the supernatant was removed by allowing the cells to spontaneously sediment. After addition of Day 8 medium for myocardial induction, the cells were incubated under 37° C. and 5% CO₂ conditions. The medium was replaced with the medium under the same conditions every 2 days.

After incubation, the resulting cells were evaluated and it was found that, in protocol 1 or 3, weak beating in some of the cell clusters or no beating was observed from Day 16 to Day 22, whereas, in protocol 2, beating was observed over the entire cell clusters and in no less than 80% of the cell clusters from Day 16 to Day 22.

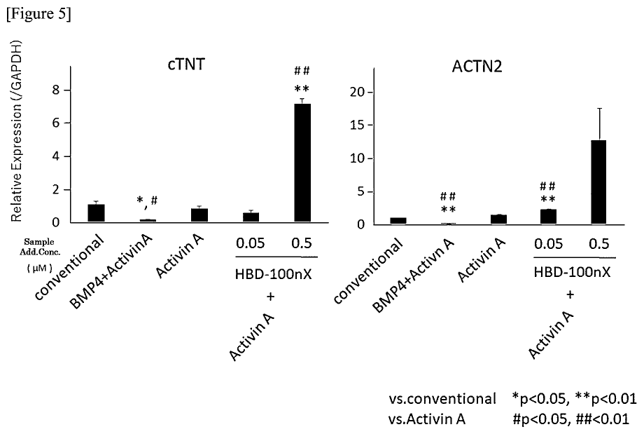

In addition, to confirm whether cardiomyocytes were differentiated, the expression levels of the cardiomyocyte markers cTNT and Actinin were examined by quantitative real-time PCR (RT-qPCR) on Day 23. Specifically, total RNA was collected by QIAGEN Rneasy Kit (QIAGEN), and cDNA was prepared by reverse transcription of RNA using the cDNA Reverse Transcription Kit (manufactured by ThermoScientific). Real-time PCR was performed on the obtained cDNA with the TaqManPCR method, using primers that specifically amplify cTNT and Actinin and probes that bind specifically to the respective genes. GAPDH levels were measured as an internal standard control. The results are shown in FIG. 5.

As shown in FIG. 5, in protocol 2 wherein the present inventive peptide (HBD-100nX) was used, the expression levels of cTNT and ACTN2 significantly increased compared to those of conventional protocol 1 (Day 1; addition of three kinds: Activin A, BMP4, and bFGF), and control protocols 3 (Day 1; addition of Activin A only) and 4 (Day 1; addition of two kinds: Activin A and BMP4). The results also showed a peptide concentration-dependent increase in gene expression.

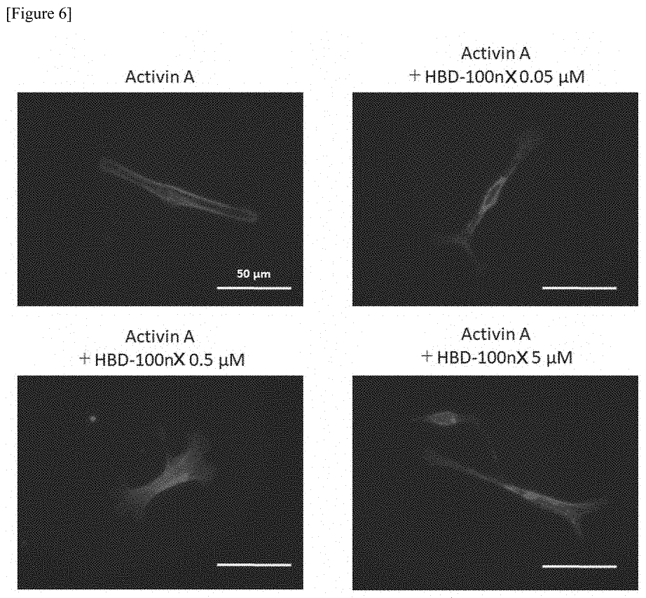

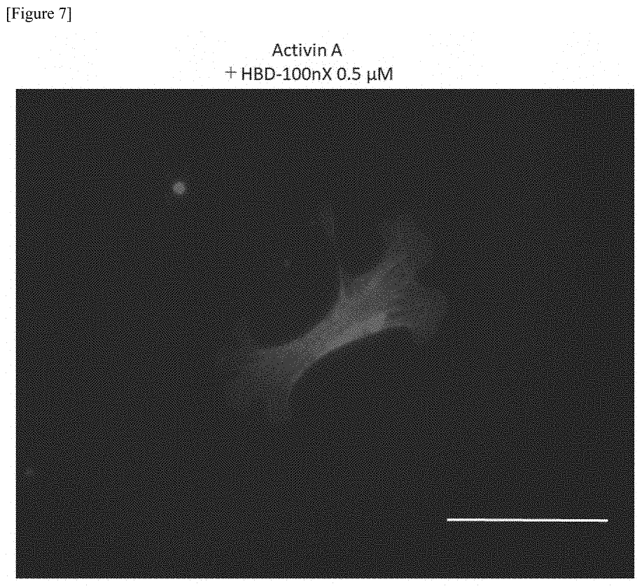

Furthermore, on Day 23 Actinin immunostaining was used to observe whether cardiomyocyte-specific sarcomere structures were recognized. Specifically, Day 23 cell clusters were reduced to single cells by collagenase and Accumax treatments, and then the cells were seeded at 10000 cells/well to 24-well plates coated with fibronectin. The next day, the cells were fixed with 4% paraformaldehyde solution and permeabilized with 0.1% Triton-X/PBS. The cells were then subjected to blocking treatment with PBS containing 5% Donkey Serum, and 1% BSA. After washing with 0.01% Triton-X/PBS, mouse anti-Actinin antibody (Sigma) was added and the reaction was carried out at room temperature for 60 minutes. After that, the cells were washed with 0.01% Triton-X/PBS, Alexa594-modified Anti-mouse antibody (abcam) was added, and the reaction was carried out at room temperature for 60 minutes. The fluorescence of the cells was observed with a Keyence BZ-X810. The results are shown in FIGS. 6 and 7.

As shown in FIGS. 6 and 7, in the protocol wherein the present inventive peptide (HBD-100nX) was used, a prominent sarcomere structure and the formation of highly functional cardiomyocytes were observed.

### [Example 3] Differentiation Induction of iPS Cells into Cardiomyocytes (Evaluation of 100nX-Dimer)

Next, a dimerized peptide of 100nX (100nX-Dimer) was evaluated. A monomer of the present inventive peptide (100nX-Monomer) was used as a control.

Differentiation induction of cardiomyocyte was performed as in Example 2, with the following changes to the medium used on Day 1. In the groups acted with peptide samples, STEMPRO34 supplemented with 1% L-Glutamine, 150 μg/mL Transferrin, 50 μg/mL Ascorbic Acid, 3.9×10⁻³% MTG, 0.05 μM 100nX-Dimer or 100nX-Monomer, and 6 ng/mL Activin A (R&D) was used (the present inventive differentiation induction method; FIG. 1, protocols 5 and 6).

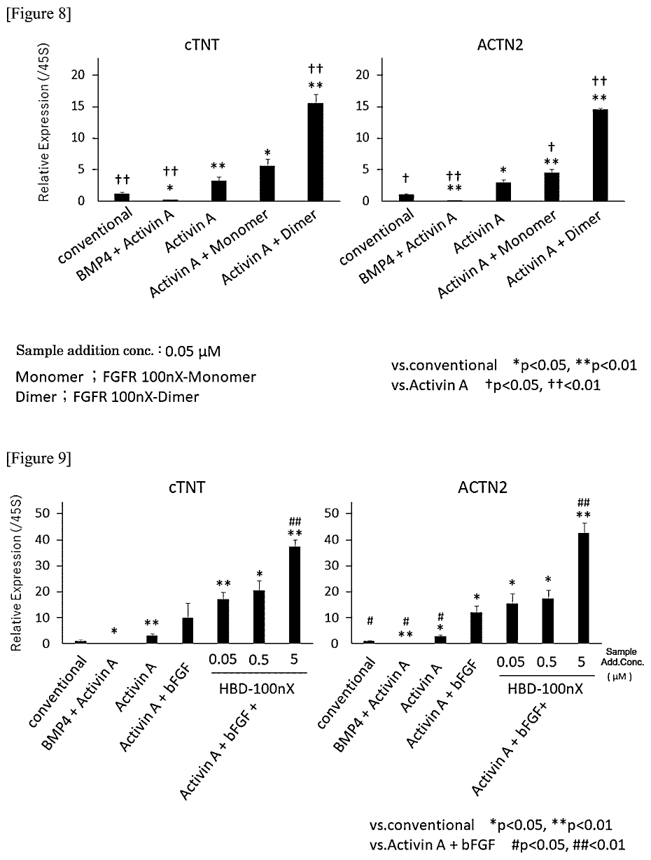

After incubation, the resulting cells were evaluated and found to have beating cell clusters on Day 11 in protocol 5, whereas, in protocol 6, beating was observed on or after Day 16. In addition, to confirm whether cardiomyocytes were differentiated, the expression levels of the cardiomyocyte markers cTNT and Actinin were examined by quantitative real-time PCR (RT-qPCR) on Day 23. Specifically, total RNA was collected by QIAGEN Rneasy Kit (QIAGEN), and cDNA was prepared by reverse transcription of RNA using the cDNA Reverse Transcription Kit (manufactured by ThermoScientific). Real-time PCR was performed on the obtained cDNA with the TaqManPCR method, using primers that specifically amplify cTNT and Actinin and probes that bind specifically to the respective genes. GAPDH levels were measured as an internal standard control. The results are shown in FIG. 8.

As shown in FIG. 8, in protocol 5 wherein the present inventive peptide (100nX-Dimer) was used, the expression levels of cTNT and ACTN2 significantly increased compared to those of conventional protocol 1 (Day 1; addition of three kinds: Activin A, BMP4, and bFGF), and control protocols 3 (Day 1; addition of Activin A only), 4 (Day 1; addition of two kinds: Activin A and BMP4), or 6.

### [Example 4] Differentiation Induction of iPS Cells into Cardiomyocytes (Evaluation of Conditions under which Peptide Samples were Mixed with bFGF)

Next, the method in which the present inventive peptide and bFGF are brought to act simultaneously was evaluated.

Differentiation induction of cardiomyocyte was performed as in Example 2, with the following changes to the medium used on Day 1. In the groups acted with peptide samples, STEMPRO34 supplemented with 1% L-Glutamine, 150 μg/mL Transferrin, 50 μg/mL Ascorbic Acid, 3.9×10⁻³% MTG, 5 ng/mL bFGF, 0.05 to 5 μM HBD-100nX or 5 μM 100nX-Dimer or 5 μM 100nX-Monomer, and 6 ng/mL Activin A (R&D) was used (the present inventive differentiation induction method; FIG. 1, protocols 7, 8 and 9). In addition, as a control group, the group in which STEMPRO 34 supplemented with 1% L-Glutamine, 150 μg/mL Transferrin, 50 μg/mL Ascorbic Acid, 3.9×10⁻³% MTG, 5 ng/mL bFGF, and 6 ng/mL Activin A (R&D) was added was prepared (FIG. 1, protocol 10).

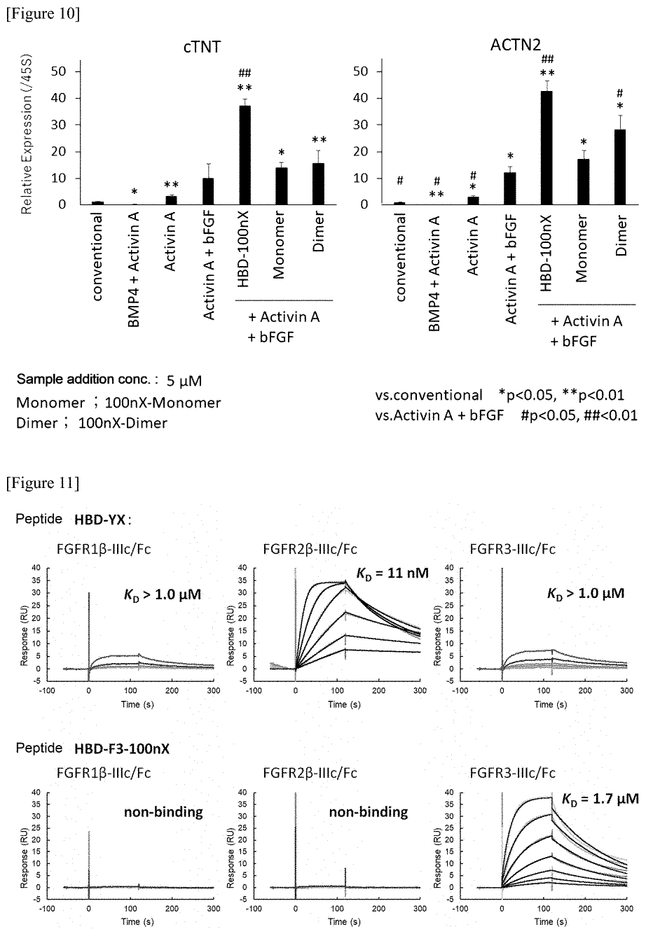

After incubation, the resulting cells were evaluated and found to have beating cell clusters on or after Day 10 to Day 11 in any of protocols 7, 8, 9, and 10, and there was no difference in the state of beating among the groups. On the other hand, to confirm whether cardiomyocytes were differentiated, the expression levels of the cardiomyocyte markers cTNT and Actinin were examined by quantitative real-time PCR (RT-qPCR) on Day 23. Specifically, total RNA was collected by QIAGEN Rneasy Kit (QIAGEN), and cDNA was prepared by reverse transcription of RNA using the cDNA Reverse Transcription Kit (manufactured by ThermoScientific). Real-time PCR was performed on the obtained cDNA with the TaqManPCR method, using primers that specifically amplify cTNT and Actinin and probes that bind specifically to the respective genes. 45S rRNA levels were measured as an internal standard control. The results are shown in FIGS. 9 and 10.

As shown in FIGS. 9 and 10, when the present inventive peptide (HBD-100nX) was used in combination with bFGF, the cTNT and ACTN2 expression levels increased dependent on peptide concentration. In addition, all of the present inventive peptides (HBD-100nX, 100nX-Dimer, and 100nX-Monomer) significantly increased the gene expression levels compared to conventional protocol 1, and HBD-100nX showed the highest value.

Therefore, use of 100nX in combination with bFGF would promote induction of differentiation into cardiomyocytes or promote maturation of cardiac myocardium.

### [Example 5] Preparation of Phage Surface-Displayed Peptide Library

First, the phagemid vector pComb3 (refer to Barbas, C. et al., Assembly of Combinatorial Antibody Libraries on Phage Surfaces: The Gene III Site, Proc. National Acad. Sci., 88, 7978-7982 (1991), and Fujii, I. et al., Evolving Catalytic Antibodies in a Phage-Displayed Combinatorial Library., Nat. Biotechnol. 16, 463-467 (1998)) was used to create six phage surface-displayed peptide libraries. The peptide chains constituting said libraries, respectively, have the amino acid sequences shown in Table 7. These peptide chains were constructed on the basis of the amino acid sequence of peptide YT-1 consisting of the known sequence (AELAALEAELAALEGGGGGGGKLAALKAKLAALKA; SEQ ID NO: 51). The amino acid X in the table represents an amino acid that is not considered to be involved in the maintenance of steric structure in the two α-helices, each consisting of 14 amino acid residues constituting the HLH (helix-loop-helix) structure, and can be substituted by an arbitrary amino acid. In addition, each peptide chain constituting the library has two peptide CA added to the N-terminus of α-helix at the N-terminal side and two peptide CA added to the C-terminus of α-helix at the C-terminal side. Note that in Table 7, ΔPTA-6R-Loop11-C is a randomized amino acid sequence in which the amino acid sequence of the loop portion of YT-1 is elongated.

TABLE 7

| Library | | Degenerate |
|---|---|---|
| Name | Library Sequence | codon |
| Δ PTA- | CAAELAALEAELAALEGGGGGGG---- | X:NDK |
| 10RC | XLXXLXXXLXXLXXAC | |
| Δ PTA- | CAXELXALXXELXALXGGGGGGG---- | X:NDK |
| 12RC-1 | KLXALXXKLXALXXAC | |
| Δ PTA- | CAXXLAXLEXXLAXLEGGGGGGG---- | X:NDK |
| 12RC-2 | KLXALXXKLXALXXAC | |
| Δ PTA- | CAXXLXXLXXXLXXLXGGGGGGG---- | X:NDK |
| 20RC | XLXXLXXXLXXLXXAC | |
| Δ PTA- | CAAELAALEAELAALEGXXXXXXXXXG | X:NNK, |
| 6R- | KLZZLKZKLZZLKZAC | Z:NDK |
| Loop11- | | |
| C | | |
| MFLIV- | CAAXZAXZEAXZAXZEGGGGGGG---- | X:NDK, |
| 8R-Δ | XZAXZKAXZAXZKAAC | Z:DTS |
| PTA- | | |
| 8RC-1 | | |
| MFLIV- | CAAEZXAZXAEZXAZXGGGGGGG---- | X:NDK, |
| 8R-Δ | KZXAZXAKZXAZXAAC | Z:DTS |
| PTA- | | |
| 8RC-2 | | |

### [Example 6] Preparation of Yeast Surface-Displayed Peptide Library

The yeast expression plasmid pYD11-BxXN (refer to Ramanayake Mudiyanselage T. M. R. et al., An Immune-Stimulatory Helix-Loop-Helix Peptide: Selective Inhibition of CTLA-4-B7 Interaction, ACS Chem. Biol., 15, 360-368 (2020)) was used to create six yeast surface-displayed peptide libraries. The peptide chains constituting said libraries, respectively, have the amino acid sequences shown in Table 8. These peptide chains were constructed on the basis of the amino acid sequence of peptide YT-1 consisting of the known sequence (AELAALEAELAALEGGGGGGGKLAALKAKLAALKA; SEQ ID NO: 51). The amino acid X in the table represents an amino acid that is not considered to be involved in the maintenance of steric structure in the two α-helices, each consisting of 14 amino acid residues constituting the HLH (helix-loop-helix) structure, and is an arbitrary amino acid designated by three bases of any of NDK, NNK, or BNS. In addition, each peptide chain constituting the library has two peptide CA added to the N-terminus of α-helix at the N-terminal side and two peptide CA added to the C-terminus of α-helix at the C-terminal side. Note that in Table 7, ΔPTA-6R-Loop11-C, ΔIKMNT-Loop11-C, and ΔPTA-6R-ΔIKMNT-Loop11-C are randomized amino acid sequences in which the amino acid sequence of the loop portion of YT-1 is elongated.

TABLE 8

| | | | Degen- |
|---|---|---|---|
| | Library | | erate |
| Library | size | Sequence | codon |
| Δ PTA- | 1.2 × 10⁷ | CAAELAALEAELAALEGGGGGG | X:NDK |
| 10RC | | G----XLXXLXXXLXXLXXAC | |
| Δ PTA- | 3.2 × 10⁸ | CAAELAALEAELAALEGXXXXX | X:NNK, |
| 6R- | | XXXXGKLZZLKZKLZZLKZAC | Z:NDK |
| Loop11- | | | |
| C | | | |
| Δ | 1.2 × 10⁷ | CAAELAALEAELAALEGXXXXX | X:BNS |
| IKMNT- | | XXXXGKLAALKAKLAALKAAC | |
| Loop11- | | | |
| C | | | |
| Δ PTA- | 2.0 × 10⁶ | CAAELAALEAELAALEGXXXXX | X:BNS, |
| 6R-Δ | | XXXXGKLZZLKZKLZZLKZAC | Z:NDK |
| IKMNT- | | | |
| Loop11C | | | |
| Δ PTA- | 3.2 × 10⁸ | CAXELXALXXELXALXGGGGGG | X:NDK |
| 12RC-1 | | G----KLXALXXKLXALXXAC | |
| Δ PTA- | 3.0 × 10⁸ | CAXXLAXLEXXLAXLEGGGGGG | X:NDK |
| 12RC-2 | | G----KLXALXXKLXALXXAC | |

### [Example 7] Biopanning for Obtaining FGFR3 Binding Peptide

The phage surface-displayed libraries obtained in Example 5 was mixed, and biopanning against FGFR3 (FGF receptor 3)-Fc chimeras was performed. First, as a negative selection, 10¹² cfu of peptide-presenting phages in 200 μl of PBS (pH 7.4) were acted on a plate immobilized with hIgG Fc and phages that did not bind to hIgG Fc were collected. The collected phage library was mixed with 100 nM of FGFR3 solution and allowed to react overnight at 4° C. (Table 9: Panning Round 1). After the reaction, the binding phages were captured by magnetic beads modified with protein A, and washed three times with PBS-T, followed by eluting the bound phages with Gly-HCl (pH 2.0). After neutralizing with 2M Tris, the pages were infected to E. coli (TG1 strain). After 4 hours of incubation, helper phages were added, and the E. coli (TG1 strain) were allowed to produce phages overnight. Next, phage solution was prepared from the culture supernatant. The prepared phage solution was mixed with FGFR3 solution and allowed to react overnight at 4° C. (Table 9: Panning Round 2). After the reaction, the bound phages were eluted. Phage solution prepared in the same way was mixed with FGFR3 solution and allowed to react at 4° C. for 1 hour (Table 9: Panning Round 3). After the reaction, the bound phages were eluted. This operation was further repeated, and after Panning Round 4, the bound phages were eluted. Note that as the rounds proceeded, the number of washes with PBS-T was increased. (Table 9). The titer of the Output phage in each round was also determined. The titer of phage was determined from the number of colonies obtained by incubating the E. coli TG1 strain contacted with the phage at 37° C. overnight. The results are shown on the right side of Table 9.

TABLE 9

| Panning | Target | Negative- | Incubation | Washing | | Output |
|---|---|---|---|---|---|---|
| Round | conc. | Selection | time | condition | Elution | phage |
| 1 | 100 nM | 10 μg/mL hIgG Fc | Overnight | PBST 3 | Gly-HCl, pH 2.0 | 6.4 × 10⁶ |
| | | 1 hr at R.T. | at 4° C. | times | 10 min at R.T. | |
| 2 | 100 nM | 10 μg/mL hIgG Fc | Overnight | PBST 5 | Gly-HCl, pH 2.0 | 1.5 × 10⁶ |
| | | 1 hr at R.T. | at 4° C. | times | 10 min at R.T. | |
| 3 | 100 nM | 10 μg/mL hIgG Fc | 60 min | PBST 10 | Gly-HCl, pH 2.0 | 6.3 × 10⁷ |
| | | 1 hr at R.T. | at 4° C. | times | 10 min at R.T. | |
| 4 | 100 nM | 10 μg/mL hIgG Fc | 30 min | PBST 15 | Gly-HCl, pH 2.0 | 7.7 × 10⁷ |
| | | 1 hr at R.T. | at 4° C. | times | 10 min at R.T. | |

### [Example 8] Biopanning for Obtaining FGFR2 Binding Peptide

Yeast clones binding to FGFR2 (FGF receptor 2) were screened by magnetic-activated cell sorting (MACS) from the yeast surface-displayed library obtained in Example 5. First, after the yeast surface-displayed peptide library was mixed with FGFR2-Fc fused protein, protein A-labeled magnetic beads which binds to the Fc moiety were added. Cells were then charged to an LS column attached to a magnetic stand. In order to remove yeast cells not bound to FGFR2, 7 mL of PBSM was flowed through the LS column twice. The LS column was then removed from the magnetic stand and FGFR2-binding yeast clones were collected by flowing SDCAA medium and cultured. The above operations were deemed as one round, and a total of three rounds were performed (Table 10). Next, yeast clones with high binding activity were screened from the yeast collected by MACS, using FACS (Fluorescence-activated cell sorting). FGFR2-Fc and mouse anti-FLAG antibodies were bound to the yeast library, followed by binding of anti-mouse IgG-Alexa488 and anti-human Fc antibody-Alexa647. Since a FLAG tag is expressed at the C-terminus of the HLH peptide, the displayed amount of peptide can be detected from the fluorescence intensity of Alexa488. The binding of the peptide to FGFR2 can also be detected by the fluorescence intensity of Alexa647. With that, yeast clones with high fluorescence intensities of Alexa488 and Alexa647 were collected by FACS cell sorter (BD FACS AriaIII).

TABLE 10

| Panning | | | Washing |
|---|---|---|---|
| Round | Target conc. | Incubation time | condition |
| 1 (MACS) | 100 nM | 60 min at 4° C. | 7 mL PBSM 2 times |
| 2 (MACS) | 100 nM | 60 min at 4° C. | 7 mL PBSM 2 times |
| 3 (MACS) | 100 nM | 60 min at RT | 7 mL PBSM 2 times |
| 4 (FACS) | 100 nM | 60 min at RT | — |

### [Example 9] Determination of Amino Acid Sequence

The phage library eluted after panning of Example 7 (Table 9: Input phage (Round 4)) was subjected to Human IgG Fc-immobilized plate, and negative selection was performed by collecting unbound phages. The phage library was then acted to FGFR3 prepared to concentrations of 100 nM, 10 nM, and 1 nM. Bound phages were then collected. The genomic DNA of the collected phages was extracted, samples were prepared according to Illumina's protocol for Miseq, and a comprehensive analysis of the phage genomic DNA was performed. Among the sequences obtained, 20 sequences high incidence rates were selected (Table 11). The yeast cells acquired in the screening of Example 8 were seeded onto SDCAA agar plates and cultured. Fourteen single colonies formed on the agar plates were randomly selected, and for each clone, the peptide DNA sequence incorporated into the yeast expression plasmid was analyzed using the Sanger method (Table 12).

TABLE 11

| | | | 1 nM | 10 nM | 100 nM | | | |
|---|---|---|---|---|---|---|---|---|
| SEQ | | | Num. of | Num. of | Num. of | 1 nM | 10 nM | 100 nM |
| No. | ID | Library | reads | reads | reads | Ranking | Ranking | Ranking |
| 27 | F3-100n9 | Uncorrected | 3326 | 2152 | 2566 | 1 | 3 | 4 |
| 28 | F3-100n251 | dPTA-6R-Loop11C | 3203 | 2387 | 3153 | 2 | 1 | 2 |
| 29 | F3-100n131 | dPTA-6R-Loop11C | 2801 | 2079 | 2648 | 3 | 4 | 3 |
| 30 | F3-100n410 | dPTA-6R-Loop11C | 2547 | 1717 | 2279 | 4 | 5 | 5 |
| 31 | F3-100n228 | dPTA-6R-Loop11C | 2006 | 1447 | 1753 | 5 | 6 | 7 |
| 32 | F3-100n116 | Uncorrected | 1895 | 2291 | 3236 | 6 | 2 | 1 |
| 33 | F3-100n182 | dPTA-6R-Loop11C | 1723 | 1143 | 1423 | 7 | 8 | 8 |
| 34 | F3-100n174 | Uncorrected | 1668 | 1223 | 1811 | 8 | 7 | 6 |
| 35 | F3-100n117 | dPTA-6R-Loop11C | 1355 | 948 | 1188 | 9 | 9 | 12 |
| 36 | F3-100n458 | dPTA-6R-Loop11C | 1304 | 789 | 1023 | 10 | 17 | 20 |
| 37 | F3-100n548 | dPTA-6R-Loop11C | 1248 | 873 | 1088 | 11 | 13 | 17 |
| 38 | F3-100n322 | dPTA-6R-Loop11C | 1234 | 813 | 1061 | 12 | 16 | 18 |
| 39 | F3-100n386 | dPTA-6R-Loop11C | 1216 | 781 | 1028 | 13 | 18 | 19 |
| 40 | F3-100n50 | Uncorrected | 1149 | 820 | 1121 | 14 | 15 | 14 |
| 41 | F3-100n192 | dPTA-6R-Loop11C | 1100 | 707 | 857 | 15 | 23 | 28 |
| 42 | F3-100n16 | dPTA-6R-Loop11C | 1095 | 647 | 775 | 16 | 26 | 33 |
| 43 | F3-100n3 | dPTA-6R-Loop11C | 1084 | 613 | 850 | 17 | 29 | 29 |
| 44 | F3-100n504 | dPTA-6R-Loop11C | 1062 | 741 | 1006 | 18 | 21 | 21 |
| 45 | F3-100n161 | Uncorrected | 1045 | 903 | 1261 | 19 | 10 | 9 |
| 46 | F3-100n250 | dPTA-6R-Loop11C | 966 | 856 | 1096 | 20 | 14 | 16 |

TABLE 12

| | | Fre- | |
|---|---|---|---|
| Clone | Amino acid sequence | quency | Library |
| FGFR_ | CAAELAALEAELAALEGGGGGG | 1 | Δ PTA- |
| Y01 | G----KLMDLHYQLYNLFIAC | | 10RC |
| FGFR_ | CARELQALLKELQALGGGGGGG | 1 | Δ PTA- |
| Y02 | G----KLLALFVKLYALRLAC | | 12RC-1 |
| FGFR_ | CAQELVALHMELRALGGGGGGG | 1 | Δ PTA- |
| Y04 | G----KLQALYLKLFALDHAC | | 12RC-1 |
| FGFR_ | CAVELYALHRELYALYGGGGGG | 1 | Δ PTA- |
| Y05 | S----KLYALYLKLLALQLAC | | 12RC-1 |
| FGFR_ | CARELRALRRELRALQGGGGGG | 1 | Δ PTA- |
| Y06 | G----KLHALHRKLFALMVAC | | 12RC-1 |
| FGFR_ | CAAELAALEAELAALEGGGGGG | 1 | Δ PTA- |
| Y07 | G----FLRNLHIKLYLLQFAC | | 10RC |
| FGFR_ | CALELLALRGELEALRGGGGGG | 1 | Δ PTA- |
| Y08 | G----KLGALHLKLFALQWAC | | 12RC-1 |
| FGFR_ | CAAELAALEAELAALEGPPRPH | 1 | Δ PTA- |
| Y09 | LPPLGKLFLLKFKLYLLKGAC | | 6R- |
| | | | Loop11- |
| | | | C |
| FGFR_ | CARELQALGKELKALKGGGGGG | 1 | Δ PTA- |
| Y10 | G----KLRALQLKLYALQVAC | | 12RC-1 |
| FGFR_ | CAKELLALKRELKALQGGGGGG | 2 | Δ PTA- |
| Y11 | G----KLRALQLKLYALLLAC | | 12RC-1 |
| FGFR_ | CAAELAALEAELAALEGPSRVG | 1 | Δ PTA- |
| Y13 | SRRRGKLNLLKYKLHGLKLAC | | 6R- |
| | | | Loop11- |
| | | | C |
| FGFR_ | CPRELEALRRELGALMGGGGGG | 1 | Δ PTA- |
| Y14 | G----KLNALMLKLYALHNAC | | 12RC-1 |
| FGFR_ | CAQELRALLRELQALGGGGGGG | 1 | Δ PTA- |
| Y15 | G----KLGALVFKLYALMLAC | | 12RC-1 |
| FGFR_ | CAHHLASLEYDLAILEGGDGGG | 1 | Δ PTA- |
| Y16 | G----HLARLKRQLAELKNAC | | 12RC-2 |

### [Example 10] Synthesis of Peptide and Confirmation of Binding to the FGF Receptor

One clone was selected from the amino acid sequences of the clones obtained in Example 8, and the peptide (F3-100nX) was synthesized based on the Fmoc solid-phase synthesis method. According to the operation manual of the SPR instrument Biacore T200 (BIACORE, Inc.), FGFR1, FGFR2, and FGFR3 were immobilized as ligands to the instrument's sensor chip CM5 using the amine coupling method. Ethanolamine was immobilized as a reference, and the peptides were measured as analyte. All measurements were performed at 25° C. and TBS was used as the running buffer. After injecting the running buffer dissolving the peptide into the CM5 sensor chip immobilized with various FGFRs (flow rate 30 μl/min, 2 min), the peptide was dissociated by flowing the running buffer for 3 min. Dissociation constants were calculated by kinetic analysis using Biacore T200 Evaluation Software (BIACORE, Inc.). The obtained sensorgram was directly curve-fitted with the reaction equation, and the rate constant was calculated by a nonlinear least-squares method. A 1:1 binding model was used in the analysis. The results are shown in FIG. 11.

### [Example 11] Peptides Binding to FGFR1 and FGFR3 (100nX)

100nX, the same peptides as in Example 1 were prepared and used in the experiment (Table 6).

### [Example 12] Differentiation Induction of iPS Cells into Cardiomyocytes (Evaluation of HBD-100nX, HBD-YX, and HBD-F3-100nX)

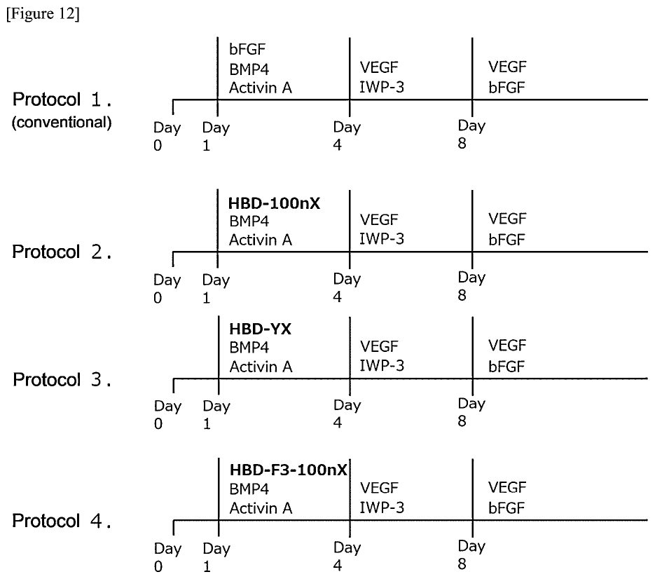

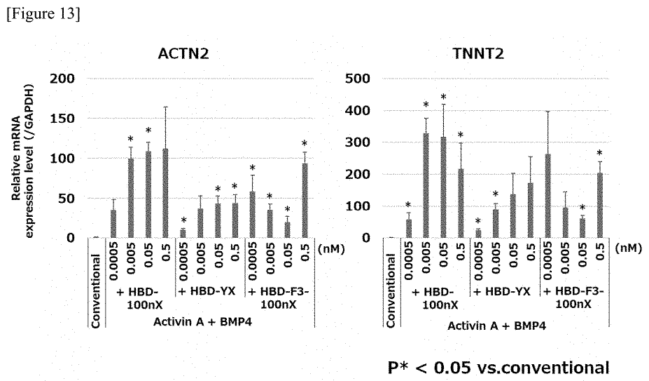

The heparan sulfate binding peptides WQPPRARIG (SEQ ID NO: 49) fused to 100nX, YX, or F3-100nX (HBD-100nX, HBD-YX, and HBD-F3-100nX) were synthesized and applied to a system for inducing differentiation of iPS cells into cardiomyocytes. The used iPS cells were the “iPS cell-KAC, hFB(N)” (KAC, product number; IPS-F001) (hereinafter referred to as “iPS-hFB”). First of all, at the start of differentiation induction (Day 0), iPS-hFBs seeded into 100φ dishes on 7 days before the start of differentiation induction (Day-7) were washed with 4 mL of PBS. Three mL of 0.5xTrypLE Select was added, and allowed to stand for 3 minutes at 37° C. After confirming that the cells had become spherical, 0.5xTrypLE Select was removed. The cells were further washed with 4 mL of PBS and 4 mL of differentiation induction medium (STEMPRO34 (Invitrogen) supplemented with 1% L-Glutamine (Invitrogen), 150 μg/mL Transferrin (Roche), 50 μg/mL Ascorbic Acid (sigma), 3.9×10⁻³% MTG (1-Thyoglycerol) (sigma), 10 μM Rock inhibitor (Y-27632, Wako), 5% matrigel (Corning), and 2 ng/mL BMP4 (R&D)) was added. Cells were then detached with a cell scraper and isolated by pipetting. The number of cells were measured, and the cells were seeded into 96-well round-bottom plates at 8000 cells/well and incubated under 37° C. and 5% CO₂ conditions. The next day (Day 1), STEMPRO34 supplemented with 1% L-Glutamine, 150 μg/mL Transferrin, 50 μg/mL Ascorbic Acid, 3.9×10⁻³% MTG, 10 ng/mL BMP4, 5 ng/mL bFGF (R&D) and 6 ng/mL Activin A (R&D) was added at 100 μl/well (conventional protocol; FIG. 12, Protocol 1). On the other hand, in the groups acted with peptide samples, STEMPRO34 supplemented with 1% L-Glutamine, 150 μg/mL Transferrin, 50 μg/mL Ascorbic Acid, 3.9×10⁻³% MTG, 10 ng/mL BMP4, 0.0005, 0.005, 0.05 or 0.5 μM HBD-100nX, HBD-YX, and HBD-F3-100n9 (SEQ ID NO: 27), and 6 ng/mL Activin A (R&D) was added at 100 μl/well (the present inventive protocol; FIG. 12, protocols 2, 3, and 4). The cells were then incubated under 37° C. and 5% CO₂ conditions for 3 days. Subsequently (Day 4), the resulting EBs were centrifuged at 200 g for 3 minutes to remove the medium. PBS was added and the supernatant was removed by centrifuging at 200 g for 3 minutes. After Accumax was added and allowed to stand at 37° C. for 5 minutes, single cells were dissociated by pipetting, 2 mL of STEMPRO34 (Day 4 medium for myocardial induction) supplemented with 1% L-Glutamine, 150 μg/mL Transferrin, 50 μg/mL Ascorbic Acid, 3.9×10⁻³% MTG, 10 ng/mL VEGF and 1 μM IWP-3 was added, and the supernatant was removed by centrifuging at 200 g for 3 minutes. The cells were suspended in Day 4 medium for myocardial induction, and after the number of cells was measured, the cells were seeded in 96-well round-bottom plates at 10000 cells/well. The cells were then incubated under 37° C. and 5% CO₂ conditions for 4 days. Subsequently (Day 8), the cell clusters were transferred in batches from 96-well plates to 24-well plates (no more than 8 cell clusters per well). After they were allowed to spontaneously sediment, the medium was removed. Then, STEMPRO34 (Day 8 medium for myocardial induction) supplemented with 1% L-Glutamine, 150 μg/mL Transferrin, 50 μg/mL Ascorbic Acid, 3.9×10⁻³% MTG, 10 ng/mL VEGF and 5 ng/mL bFGF was added, and the supernatant was removed by allowing the cells to spontaneously sediment. After addition of Day 8 medium for myocardial induction, the cells were incubated under 37° C. and 5% CO₂ conditions. The medium was replaced with the medium under the same conditions every 2 days. After incubation, to confirm whether cardiomyocytes were differentiated, the expression levels of the cardiomyocyte markers cTNT and Actinin were examined by quantitative real-time PCR (RT-qPCR) on Day 23 (FIG. 13). Specifically, total RNA was collected by QIAGEN Rneasy Kit (QIAGEN), and cDNA was prepared by reverse transcription of RNA using the cDNA Reverse Transcription Kit (manufactured by ThermoScientific). Real-time PCR was performed on the obtained cDNA with the TaqManPCR method, using primers that specifically amplify cTNT and Actinin and probes that bind specifically to the respective genes. GAPDH levels were measured as an internal standard control. The results showed that in protocols 2, 3, and 4 wherein the present inventive peptides (HBD-100nX, HBD-YX, and HBD-F3-100n9 (SEQ ID NO: 27)) were used, the expression levels of cTNT and ACTN2 significantly increased compared to those of conventional protocol 1 (Day 1; addition of three kinds: Activin A, BMP4, and bFGF), though differences were observed in the concentrations at which the effect was exerted depending on the kind of peptide. HBD-100nX and HBD-YX were shown to increase the gene expressions in a peptide concentration-dependent manner. HBD-F3-100n9 (SEQ ID NO: 27) showed a different response tendency as compared to the conditions that other peptides were added.

### [Example 13] Differentiation Induction of iPS Cells into Hepatocytes (Evaluation of 100nX)

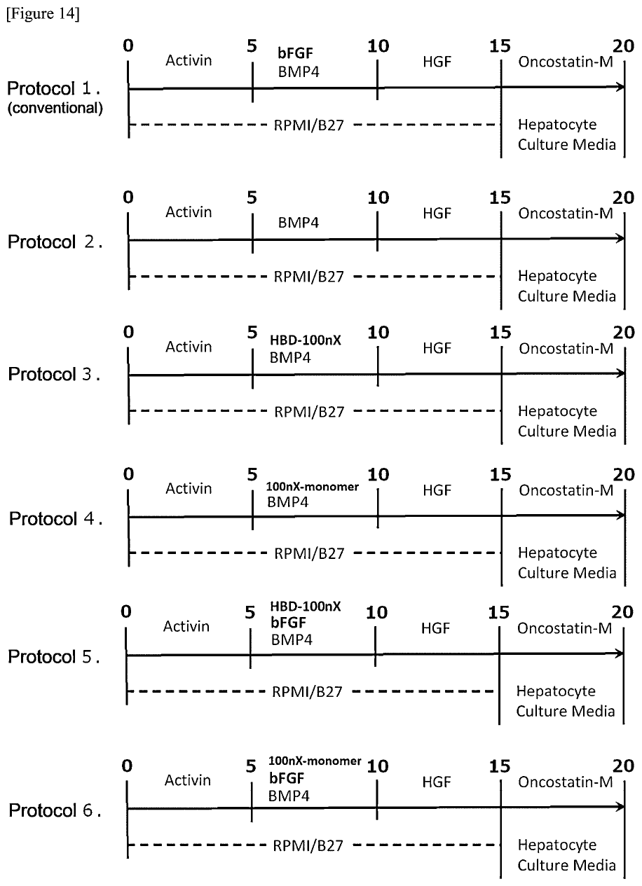

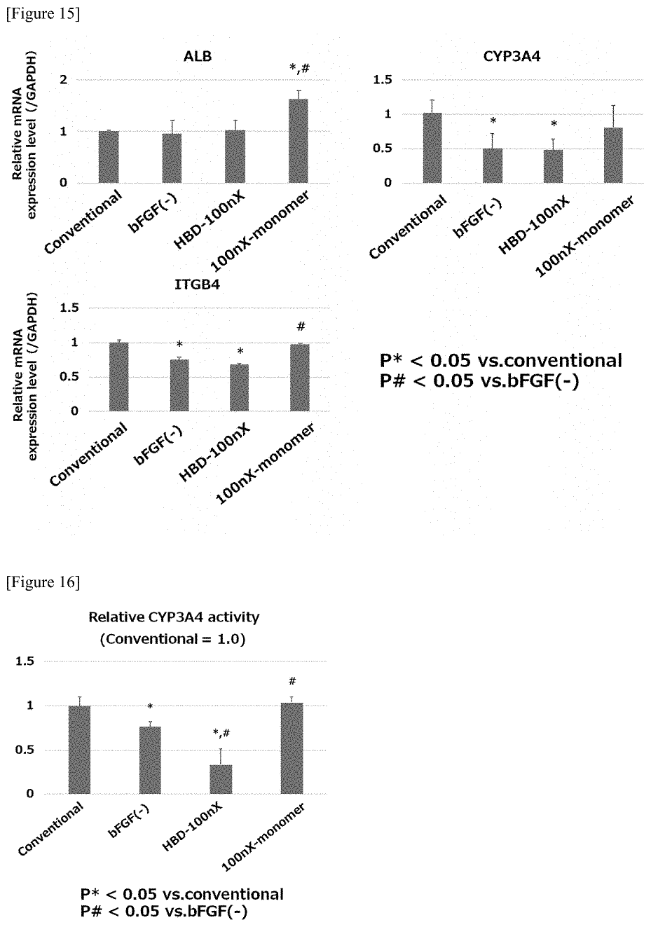

An attempt to apply 100nX to a differentiation induction system from iPS cells to hepatocytes was made. The used iPS cells were the “iPS cell-KAC, hFB(N)” (KAC, product number; IPS-F001) (hereinafter referred to as “iPS-hFB”). The iPS-hFBs seeded into 100φ dishes on 7 days before the start of differentiation induction (Day-7) were washed with 4 mL of PBS. Three ml of 0.5xTrypLE Select was added, and allowed to stand for 3 minutes at 37° C. After confirming that the cells had become spherical, 0.5xTrypLE Select was removed. The cells were further washed with 4 mL of PBS, and after 4 mL of StemFit was added, the cells were detached with a cell scraper and isolated into single cells by pipetting. Then, cells were seeded in 6-well plates at 1.5×10⁵ cells/well. On the starting day of differentiation induction (Day 0), the culture medium was removed, and differentiation induction medium (RPMI1640 (Thermo) supplemented with B-27 Plus Supplement (Thermo) and 100 ng/mL Activin A (R&D)) was added at 2 mL/wells. The medium was replaced on Day 3 using the same medium. The medium was removed on Day 5 and RPMI1640 supplemented with B-27 Plus Supplement (Thermo), 20 ng/mL BMP4, and 20 ng/mL bFGF was added at 2 mL/well (conventional protocol; FIG. 14, protocol 1). On the other hand, in the groups acted with peptide samples RPMI1640 supplemented with B-27 Plus Supplement (Thermo), 20 ng/mL BMP4, and 0.1 nM HBD-100nX or 100nX-monomer was added at 2 mL/well (conventional protocol; FIG. 14, protocols 3 and 4). Further, as a control group, RPMI1640 supplemented with B-27 Plus Supplement (Thermo), and 20 ng/mL BMP4 was added at 2 mL/well (conventional protocol; FIG. 14, protocol 2). The medium was replaced on Day 8 using the same medium. In all conditions, the medium was removed on Day 10, and RPMI1640 supplemented with B-27 Plus Supplement (Thermo), and 20 ng/mL HGF was added at 2 mL/well. The medium was replaced on Day 12 and Day 14 using the same medium. The medium was exchanged with HCM BulletKit (Lonza) (containing HBM, Ascorbic Acid, BSA-FAF, Hydrocortisone, Transferin, GA-1000, and Insulin) on Day 15 at 2 mL/well. On Day 17 and Day 18, the medium was exchanged with the same medium. After incubation, to confirm whether hepatocytes were differentiated, the expression levels of the liver markers Albumin (ALB) and CYP3A4, and those of the bile duct marker ITGB4 were examined by quantitative real-time PCR (RT-qPCR) on Day 20 (FIG. 15). Specifically, total RNA was collected by QIAGEN Rneasy Kit (QIAGEN), and cDNA was prepared by reverse transcription of RNA using the cDNA Reverse Transcription Kit (manufactured by ThermoScientific). Real-time PCR was performed on the obtained cDNA with the TaqManPCR method, using primers that specifically amplify ALB, CYP3A4 and ITGB4, and probes that bind specifically to the respective genes. GAPDH levels were measured as an internal standard control. The results showed that in protocol 4 wherein the present inventive peptide (100nX-monomer) was used, the expression level of ALB significantly increased compared to that of conventional protocol 1 (Day 5; addition of two kinds: BMP4, and bFGF), and that of control protocol 2 (Day 5; addition of BMP4). In addition, the gene expression levels of CYP3A4 and ITGB4 significantly increased compared to those of control protocol 2 (Day 5; addition of BMP4), and were similar to those of conventional protocol 1 (Day 5; addition of two kinds: BMP4, and bFGF). These results indicated that 100nX-monomer was as effective as or better than conventional bFGF in promotion of hepatic differentiation.

### [Example 14] Functional Evaluation of Hepatocytes Induced to Differentiate Using 100nX

Differentiation of iPS cells was induced in the same manner as in Example 13. After the cells were washed with PBS on Day 20, the medium was exchanged with HCM BulletKit (Lonza) containing 3 μM luciferin-IPA at 1 mL/well. After 1 hour of reaction at 37° C., 100 μL of the supernatant was transferred to a 96-well white plate., and Luciferin Detection Reagent was added at 100 μL/well. After 20 minutes of reaction at room temperature, the luminescence was measured. The cells were also crushed and the luminescence value was corrected by quantifying proteins with TaKaRa BCA Protein Assay Kit (Takara Bio). The results showed that CYP3A4 activity significantly increased compared to that of control protocol 2 (Day 5; addition of BMP4), and was similar to that of conventional protocol 1 (Day 5; addition of two kinds: BMP4, and bFGF). These results indicated that 100nX-monomer contributes to liver function at a level comparable to that of bFGF (FIG. 16).

### [Example 15] Differentiation Induction of iPS Cells into Neurons (Evaluation of 100nX)

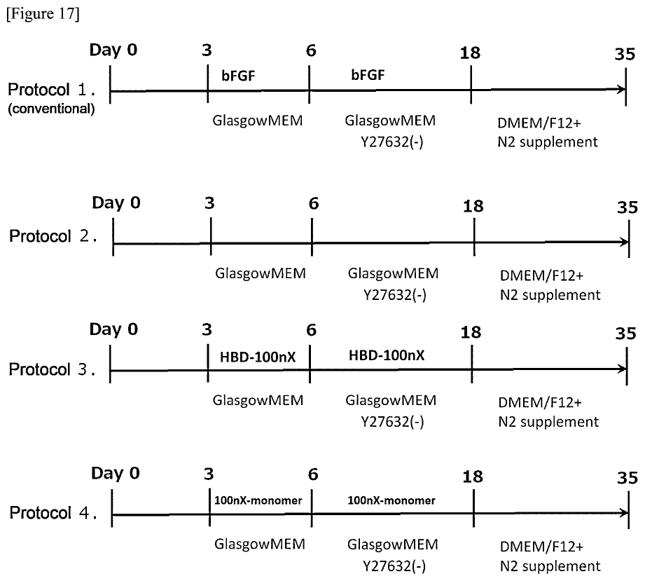

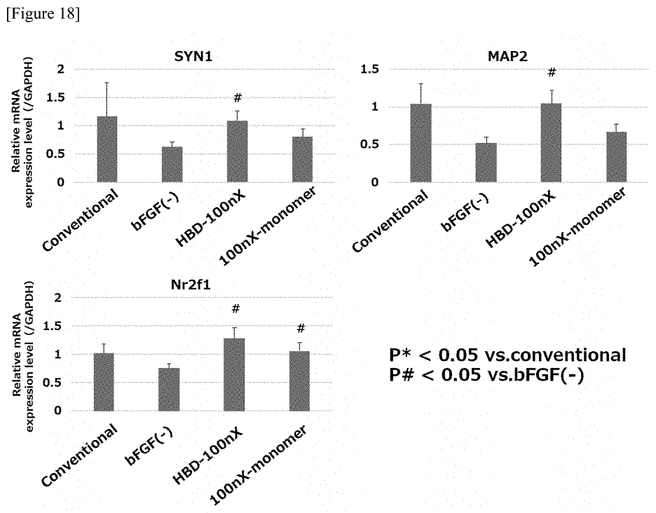

An attempt to apply 100nX to a differentiation induction system from iPS cells to neurons was made. The used iPS cells were the “iPS cell-KAC, hFB(N)” (KAC, product number; IPS-F001) (hereinafter referred to as “iPS-hFB”). First of all, at the start of differentiation induction (Day 0), iPS-hFBs seeded into 100φ dishes on 7 days before the start of differentiation induction (Day-7) were washed with 4 mL of PBS. Three ml of 0.5xTrypLE Select was added, and allowed to stand for 3 minutes at 37° C. After confirming that the cells had become spherical, 0.5xTrypLE Select was removed. The cells were further washed with 4 mL of PBS and 4 mL of differentiation induction medium (G-MEM (Thermo) supplemented with 20% KSR (Thermo), 1xNEAA (Thermo), 0.1 mM 2-mercaptoethanol (Wako Pure Chemicals), 1 mM sodium pyruvate (Thermo), 20 μM Y27632 (Wako Pure Chemicals), 3 μM IWR-1-endo (Wako Pure Chemicals), and 5 μM SB431542 (Wako Pure Chemicals)) was added. Cells were then detached with a cell scraper and isolated by pipetting. The number of cells were measured, and the cells were seeded into 96-well round-bottom plates at 9000 cells/well and incubated under 37° C. and 5% CO₂ conditions. On Day 3, G-MEM supplemented with 20% KSR (Thermo), 1xNEAA (Thermo), 0.1 mM 2-mercaptoethanol (Wako Pure Chemicals), 1 mM sodium pyruvate (Thermo), 20 μM Y27632 (Wako Pure Chemicals), 3 μM IWR-1-endo (Wako Pure Chemicals), 5 μM SB431542 (Wako Pure Chemicals), and 0.3 nM bFGF was added at 100 μl/well (conventional protocol; FIG. 17, Protocol 1). On the other hand, in the groups acted with peptide samples, G-MEM supplemented with 20% KSR (Thermo), 1xNEAA (Thermo), 0.1 mM 2-mercaptoethanol (Wako Pure Chemicals), 1 mM sodium pyruvate (Thermo), 20 μM Y27632 (Wako Pure Chemicals), 3 μM IWR-1-endo (Wako Pure Chemicals), 5 μM SB431542 (Wako Pure Chemicals), 0.25 nM HBD-100nX or 100nX-monomer was added at 100 μL/well (FIG. 14, protocols 3 and 4). A group in which G-MEM supplemented with 20% KSR (Thermo), 1xNEAA (Thermo), 0.1 mM 2-mercaptoethanol (Wako Pure Chemicals), 1 mM sodium pyruvate (Thermo), 20 μM Y27632 (Wako Pure Chemicals), 3 μM IWR-1-endo (Wako Pure Chemicals), 5 μM SB431542 (Wako Pure Chemicals) was added at 100 μL/well was prepared as a control group (FIG. 14, protocol 2). On Day 6, the entire medium was replaced with the medium of each condition containing no Y27632 (Wako Pure Chemicals). Likewise, the medium was exchanged at Day 10 and Day 14. The medium was removed on Day 18 and the cells added with DMEM/F12 (GlutaMax) (Thermo) containing 1% N-2 Supplement and 1% Chemically Defined Lipid Concentrate were transferred to EZSPHERE (AGC Technoglass) at 2 mL/well. On Day 21, 24, and 27, the medium was replaced with DMEM/F12 (GlutaMax) (Thermo) containing 1% N-2 Supplement. Further, On Day 30, and 33, the medium was replaced with DMEM/F12 (GlutaMax) (Thermo) containing 1% N-2 Supplement and 1% Chemically Defined Lipid Concentrate. After incubation, to confirm whether neurons were differentiated, the expression levels of the neuronal markers SYN1, MAP2, and Nr2f1 were examined by quantitative real-time PCR (RT-qPCR) on Day 35 (FIG. 18). Specifically, total RNA was collected by QIAGEN Rneasy Kit (QIAGEN), and cDNA was prepared by reverse transcription of RNA using the cDNA Reverse Transcription Kit (ThermoScientific). Real-time PCR was performed on the obtained cDNA with the TaqManPCR method, using primers that specifically amplify SYN1, MAP2, and Nr2f1, and probes that bind specifically to the respective genes. GAPDH levels were measured as an internal standard control. The results showed that in protocol 3 wherein the present inventive peptide (HBD-100nX) was used, the gene expression level of Nr2f1 significantly increased compared to that of conventional protocol 1 (Day 3; addition of bFGF), and that of control protocol 2 (Day 3; no addition of bFGF). The gene expression levels of SYN1 and MAP2 significantly increased compared to those of control protocol 2 (Day 3; no addition of bFGF), and were similar to those of conventional protocol 1 (Day 3; addition of bFGF). In protocol 4 wherein the present inventive peptide (100nX-monomer) was used, the expression levels of all the genes tended to increase compared to the control protocol 2 (Day 3; no addition of bFGF). Especially, the gene expression level of Nr2f1 significantly increased compared to that of control protocol 2 (Day 3; no addition of bFGF), and was similar to that of conventional protocol 1 (Day 3; addition of bFGF). These results indicated that HBD-100nX was as effective as or better than conventional bFGF in promotion of neural differentiation, and that 100nX-monomer was as effective as conventional bFGF in contribution to neural differentiation.

### INDUSTRIAL APPLICABILITY

The present invention is useful as a cell research tool for differentiating cells or promoting cell differentiation. It is also useful in tissue construction for regenerative medicine and preparation of cells for drug screening, and the like.

### SEQUENCE TABLE FREE TEXT

SEQ ID NO: 1: Xaa, the 3rd amino acid residue from the N-terminus, is Ala, Arg, Gln, Val, Leu, Lys, Phe, or His.
SEQ ID NO: 1: Xaa, the 4th amino acid residue from the N-terminus, is Glu or His.
SEQ ID NO: 1: Xaa, the 6th amino acid residue from the N-terminus, is Ala, Gln, Val, Tyr, Arg, Leu, or Glu.
SEQ ID NO: 1: Xaa, the 7th amino acid residue from the N-terminus, is Ala or Ser.
SEQ ID NO: 1: Xaa, the 9th amino acid residue from the N-terminus, is Glu, Leu, His, Arg, Gly, or Lys.
SEQ ID NO: 1: Xaa, the 10th amino acid residue from the N-terminus, is Ala, Lys, Met, Arg, Gly, or Tyr.
SEQ ID NO: 1: Xaa, the 11th amino acid residue from the N-terminus, is Glu or Asp.
SEQ ID NO: 1: Xaa, the 13th amino acid residue from the N-terminus, is Ala, Gln, Arg, Tyr, Glu, Lys, or Gly.
SEQ ID NO: 1: Xaa, the 14th amino acid residue from the N-terminus, is Ala or Ile.
SEQ ID NO: 1: Xaa, the 16th amino acid residue from the N-terminus, is Glu, Gly, Tyr, Gln, Arg, Lys, or Met.
SEQ ID NO: 1: Xaa, the 18th amino acid residue from the N-terminus, is Phe, Ala, Gln, Val, Gly, Ile, Asn, Arg, Met, Asp, Leu, Ser, Pro, Lys, or Glu.
SEQ ID NO: 1: Xaa, the 19th amino acid residue from the N-terminus, is Gly, Val, Lys, Thr, Pro, Arg, Met, Asn, Glu, Asp, Ser, Cys, Trp, His, or Ala.
SEQ ID NO: 1: Xaa, the 20th amino acid residue from the N-terminus, is Gly, Arg, Glu, Phe, Ala, Lys, Pro, Asn, Leu, Val, Met, Asp, His, Thr, or Tyr.
SEQ ID NO: 1: Xaa, the 21st amino acid residue from the N-terminus, is Val, Asp, Leu, Met, Ser, Thr, Ala, Asn, His, Gly, Phe, Glu, Arg, Pro, Tyr, or Lys.
SEQ ID NO: 1: Xaa, the 22nd amino acid residue from the N-terminus, is Val, Lys, Tyr, Thr, Asn, Gly, Asp, Glu, Pro, Phe, Gln, His, or Ile.
SEQ ID NO: 1: Xaa, the 23rd amino acid residue from the N-terminus, is Tyr, Arg, His, Leu, Pro, Asn, Glu, Met, Ala, Gly, Val, Ile, or Ser.
SEQ ID NO: 1: Xaa, the 24th amino acid residue from the N-terminus, is Ser, Lys, Met, Gly, His, Gln, Thr, Val, Cys, Leu, Asn, Ala, Glu, Pro, or Arg.
SEQ ID NO: 1: Xaa, the 25th amino acid residue from the N-terminus, is Cys, Ser, Ala, Thr, Arg, Glu, Asn, Gln, Gly, Lys, Tyr, Pro, Leu, Val, or Met.
SEQ ID NO: 1: Xaa, the 26th amino acid residue from the N-terminus, is Glu, Lys, Ser, Val, Leu, Arg, Gly, Ile, Phe, Thr, Ala, His, Gln, Asn, Met, or Tyr.
SEQ ID NO: 1: Xaa, the 27th amino acid residue from the N-terminus, is Trp or Gly.
SEQ ID NO: 1: Xaa, the 28th amino acid residue from the N-terminus, is Gln, Lys, Phe, or His.
SEQ ID NO: 1: Xaa, the 29th amino acid residue from the N-terminus, is Ala or Leu.
SEQ ID NO: 1: Xaa, the 30th amino acid residue from the N-terminus, is Val, Met, Asp, Tyr, Lys, Leu, Ile, Arg, Ser, Gln, Gly, Glu, His, Phe, Asn, or Ala.
SEQ ID NO: 1: Xaa, the 31st amino acid residue from the N-terminus, is Tyr, Met, Arg, Ser, Lys, His, Gly, Leu, Asn, Glu, Val, Asp, or Ala.
SEQ ID NO: 1: Xaa, the 33rd amino acid residue from the N-terminus, is His, Phe, Tyr, Lys, Ile, Gln, Met, or Val.
SEQ ID NO: 1: Xaa, the 34th amino acid residue from the N-terminus, is Tyr, Trp, Gly, Leu, Asn, Asp, Gln, Glu, Met, Phe, Arg, His, Ser, Lys, Val, or Ile.
SEQ ID NO: 1: Xaa, the 35th amino acid residue from the N-terminus, is Lys, Arg, or Gln.
SEQ ID NO: 1: Xaa, the 37th amino acid residue from the N-terminus, is Met, Glu, Trp, Val, His, Ser, Asn, Ile, Gln, Tyr, Gly, Leu, Arg, Phe, or Ala.
SEQ ID NO: 1: Xaa, the 38th amino acid residue from the N-terminus, is Arg, Leu, Val, Ser, His, Tyr, Phe, Asn, Lys, Gln, Trp, Glu, Ile, Gly, or Ala.
SEQ ID NO: 1: Xaa, the 40th amino acid residue from the N-terminus, is Phe, Arg, Asp, Gln, Met, Lys, Leu, or His.
SEQ ID NO: 1: Xaa, the 41st amino acid residue from the N-terminus, is Gln, Gly, Tyr, Arg, Asn, Ile, Ser, His, Glu, Met, Leu, Asp, Cys, Trp, Val, or Phe.
SEQ ID NO: 2: Xaa, the 18th amino acid residue from the N-terminus, is Phe, Ala, Gln, Val, Gly, Ile, Asn, Arg, Met, Asp, Leu, Ser, Pro, Lys, or Glu.
SEQ ID NO: 2: Xaa, the 19th amino acid residue from the N-terminus, is Gly, Val, Lys, Thr, Pro, Arg, Met, Asn, Glu, Asp, Ser, Cys, Trp, His, or Ala.
SEQ ID NO: 2: Xaa, the 20th amino acid residue from the N-terminus, is Gly, Arg, Glu, Phe, Ala, Lys, Pro, Asn, Leu, Val, Met, Asp, His, Thr, or Tyr.
SEQ ID NO: 2: Xaa, the 21st amino acid residue from the N-terminus, is Val, Asp, Leu, Met, Ser, Thr, Ala, Asn, His, Gly, Phe, Glu, Arg, Pro, Tyr, or Lys.
SEQ ID NO: 2: Xaa, the 22nd amino acid residue from the N-terminus, is Val, Lys, Tyr, Thr, Asn, Gly, Asp, Glu, Pro, Phe, Gln, His, or Ile.
SEQ ID NO: 2: Xaa, the 23rd amino acid residue from the N-terminus, is Tyr, Arg, His, Leu, Pro, Asn, Glu, Met, Ala, Gly, Val, or Ile.
SEQ ID NO: 2: Xaa, the 24th amino acid residue from the N-terminus, is Ser, Lys, Met, Gly, His, Gln, Thr, Val, Cys, Leu, Asn, Ala, Glu, or Arg.
SEQ ID NO: 2: Xaa, the 25th amino acid residue from the N-terminus, is Cys, Ser, Ala, Thr, Arg, Glu, Asn, Gln, Gly, Lys, Tyr, Pro, Leu, Val, or Met.
SEQ ID NO: 2: Xaa, the 26th amino acid residue from the N-terminus, is Glu, Lys, Ser, Val, Leu, Arg, Gly, Ile, Phe, Thr, Ala, His, Gln, Asn, Met, or Tyr.
SEQ ID NO: 2: Xaa, the 27th amino acid residue from the N-terminus, is Trp or Gly.
SEQ ID NO: 2: Xaa, the 28th amino acid residue from the N-terminus, is Gln or Lys.
SEQ ID NO: 2: Xaa, the 29th amino acid residue from the N-terminus, is Ala or Leu.
SEQ ID NO: 2: Xaa, the 30th amino acid residue from the N-terminus, is Val, Met, Asp, Tyr, Lys, Leu, Ile, Arg, Ser, Gln, Gly, or Glu.
SEQ ID NO: 2: Xaa, the 31st amino acid residue from the N-terminus, is Tyr, Met, Arg, Ser, Lys, His, Gly, Leu, Asn, Glu, or Val.
SEQ ID NO: 2: Xaa, the 34th amino acid residue from the N-terminus, is Tyr, Trp, Gly, Leu, Asn, Asp, Gln, Glu, Met, Phe, Arg, His, Ser, Lys, or Val.
SEQ ID NO: 2: Xaa, the 35th amino acid residue from the N-terminus, is Lys or Arg.
SEQ ID NO: 2: Xaa, the 37th amino acid residue from the N-terminus, is Met, Glu, Trp, Val, His, Ser, Asn, Ile, Gln, Tyr, Gly, Leu, or Arg.
SEQ ID NO: 2: Xaa, the 38th amino acid residue from the N-terminus, is Arg, Leu, Val, Ser, His, Tyr, Phe, Asn, Lys, Gln, Trp, Glu, Ile, or Gly.
SEQ ID NO: 2: Xaa, the 41st amino acid residue from the N-terminus, is Gln, Gly, Tyr, Arg, Asn, Ile, Ser, His, Glu, Met, Leu, Asp, Cys, Trp, or Val.
SEQ ID NO: 3: Xaa, the 3rd amino acid residue from the N-terminus, is Ala, Arg, Gln, Val, Leu, Lys, Phe, or His.
SEQ ID NO: 3: Xaa, the 4th amino acid residue from the N-terminus, is Glu or His.
SEQ ID NO: 3: Xaa, the 6th amino acid residue from the N-terminus, is Ala, Gln, Val, Tyr, Arg, Leu, or Glu.
SEQ ID NO: 3: Xaa, the 7th amino acid residue from the N-terminus, is Ala or Ser.
SEQ ID NO: 3: Xaa, the 9th amino acid residue from the N-terminus, is Glu, Leu, His, Arg, Gly, or Lys.
SEQ ID NO: 3: Xaa, the 10th amino acid residue from the N-terminus, is Ala, Lys, Met, Arg, Gly, or Tyr.
SEQ ID NO: 3: Xaa, the 11th amino acid residue from the N-terminus, is Glu or Asp.
SEQ ID NO: 3: Xaa, the 13th amino acid residue from the N-terminus, is Ala, Gln, Arg, Tyr, Glu, Lys, or Gly.
SEQ ID NO: 3: Xaa, the 14th amino acid residue from the N-terminus, is Ala or Ile.
SEQ ID NO: 3: Xaa, the 16th amino acid residue from the N-terminus, is Glu, Gly, Tyr, Gln, Arg, Lys, or Met.
SEQ ID NO: 3: Xaa, the 18th amino acid residue from the N-terminus, is Gly or Pro.
SEQ ID NO: 3: Xaa, the 19th amino acid residue from the N-terminus, is Gly, Pro, Asp, or Ser.
SEQ ID NO: 3: Xaa, the 20th amino acid residue from the N-terminus, is Gly or Arg.
SEQ ID NO: 3: Xaa, the 21st amino acid residue from the N-terminus, is Val, Gly, or Pro.
SEQ ID NO: 3: Xaa, the 22nd amino acid residue from the N-terminus, is Gly or His.
SEQ ID NO: 3: Xaa, the 23rd amino acid residue from the N-terminus, is Leu, Gly, or Ser.
SEQ ID NO: 3: Xaa, the 24th amino acid residue from the N-terminus, is Pro or Arg.
SEQ ID NO: 3: Xaa, the 25th amino acid residue from the N-terminus, is Arg or Pro.
SEQ ID NO: 3: Xaa, the 26th amino acid residue from the N-terminus, is Leu or Arg.
SEQ ID NO: 3: Xaa, the 27th amino acid residue from the N-terminus, is Gly.
SEQ ID NO: 3: Xaa, the 28th amino acid residue from the N-terminus, is Lys, Phe, or His.
SEQ ID NO: 3: Xaa, the 30th amino acid residue from the N-terminus, is Met, Tyr, Leu, Arg, Gln, Gly, His, Phe, Asn, or Ala.
SEQ ID NO: 3: Xaa, the 31st amino acid residue from the N-terminus, is Arg, Leu, Asn, Asp, or Ala.
SEQ ID NO: 3: Xaa, the 33rd amino acid residue from the N-terminus, is His, Phe, Tyr, Lys, Gln, Met, or Val.
SEQ ID NO: 3: Xaa, the 34th amino acid residue from the N-terminus, is Tyr, Leu, Phe, Arg, Val, or Ile.
SEQ ID NO: 3: Xaa, the 35th amino acid residue from the N-terminus, is Lys or Gln.
SEQ ID NO: 3: Xaa, the 37th amino acid residue from the N-terminus, is His, Tyr, Leu, Phe, or Ala.
SEQ ID NO: 3: Xaa, the 38th amino acid residue from the N-terminus, is Leu, Asn, Glu, Gly, or Ala.
SEQ ID NO: 3: Xaa, the 40th amino acid residue from the N-terminus, is Phe, Arg, Asp, Gln, Met, Lys, Leu, or His.
SEQ ID NO: 3: Xaa, the 41st amino acid residue from the N-terminus, is Gly, Asn, Ile, His, Leu, Trp, Val, or Phe.

1. A synthetic peptide having, in the molecular structure thereof, an amino acid sequence represented by the following (SEQ ID NO: 1), or an amino acid sequence formed by substitution, deletion and/or addition of one or several amino acid residues in the amino acid sequence:
(SEQ ID No. 1)
C A X X L X X L X X X L X X L X G X X X X X
X X X X X X X X X L X X X L X X L X X A C
• • •
in the formula, the 1st X from the N-terminus of SEQ ID NO: 1 is A, R, Q, V, L, K, F, or H, the 2nd X is E or H, the 3rd X is A, Q, V, Y, R, L, or E, the 4th X is A or S, the 5th X is E, L, H, R, G, or K, the 6th X is A, K, M, R, G, or Y, the 7th X is E or D, the 8th X is A, Q, R, Y, E, K, or G, the 9th X is A or I, the 10th X is E, G, Y, Q, R, K, or M, the 11th X is F, A, Q, V, G, I, N, R, M, D, L, S, P, K, or E, the 12th X is G, V, K, T, P, R, M, N, E, D, S, C, W, H, or A, the 13th X is G, R, E, F, A, K P, N, L, V, M, D, H, T, or Y, the 14th X is V, D, L, M, S, T, A, N, H, G, F, E, R, P, Y, or K, the 15th X is V, K, Y, T, N, G, D, E, P, F, Q, H, or I, the 16th X is Y, R, H, L, P, N, E, M, A, G, V, I, or S, the 17th X is S, K, M, G, H, Q, T, V, C, L, N, A, E, P, or R, the 18th X is C, S, A, T, R, E, N, Q, G, K, Y, P, L, V, or M, the 19th X is E, K, S, V, L, R, G, I, F, T, A, H, Q, N, M, or Y, the 20th X is W or G, the 21st X is Q, K, F, or H, the 22nd X is A or L, the 23rd X is V, M, D, Y, K, L, I, R, S, Q, G, E, H, F, N, or A, the 24th X is Y, M, R, S, K, H, G, L, N, E, V, D, or A, the 25th X is H, F, Y, K, I, Q, M, or V, the 26th X is Y, W, G, L, N, D, Q, E, M, F, R, H, S, K, V, or I, the 27th X is K, R, or Q, the 28th X is M, E, W, V, H, S, N, I, Q, Y, G, L, R, F, or A, the 29th X is R, L, V, S, H, Y, F, N, K, Q, W, E, I, G, or A, the 30th X is F, R, D, Q, M, K, L, or H, and the 31st X is Q, G, Y, R, N, I, S, H, E, M, L, D, C, W, V, or F.

2. The synthetic peptide according to claim 1, having, in the molecular structure thereof, an amino acid sequence represented by the following (SEQ ID NO: 2), or an amino acid sequence formed by substitution, deletion and/or addition of one or several amino acid residues in the amino acid sequence:
(SEQ ID No. 2)
C A A E L A A L E A E L A A L E G X X X X X
X X X X X X X X X L K X X L X X L K X A C
• • •
in the formula, the 1st X from the N-terminus of SEQ ID NO: 2 is F, A, Q, V, G, I, N, R, M, D, L, S, P, K, or E, the 2nd X is G, V, K, T, P, R, M, N, E, D, S, C, W, H, or A, the 3rd X is G, R, E, F, A, K, P, N, L, V, M, D, H, T, or Y, the 4th X is V, D, L, M, S, T, A, N, H, G, F, E, R, P, Y, or K, the 5th X is V, K, Y, T, N, G, D, E, P, F, Q, H, or I, the 6th X is Y, R, H, L, P, N, E, M, A, G, V, or I, the 7th X is S, K, M, G, H, Q, T, V, C, L, N, A, E, or R, the 8th X is C, S, A, T, R, E, N, Q, G, K, Y, P, L, V, or M, the 9th X is E, K, S, V, L, R, G, I, F, T, A, H, Q, N, M, or Y, the 10th X is W or G, the 11th X is Q or K, the 12th X is A or L, the 13th X is V, M, D, Y, K, L, I, R, S Q, G, or E, the 14th X is Y, M, R, S, K, H, G, L, N, E, or V, the 15th X is Y, W, G, L, N, D, Q, E, M, F, R, H, S, K, or V, the 16th X is K or R, the 17th X is M, E, W, V, H, S, N, I, Q, Y, G, L, or R, the 18th X is R, L, V, S, H, Y, F, N, K, Q, W, E, I, or G, and the 19th X is Q, G, Y, R, N, I, S, H, E, M, L, D, C, W, or V.

3. The synthetic peptide according to claim 2, wherein the 1st X from the N-terminus of said amino acid sequence (SEQ ID NO: 2) is F, A, Q, V, G, I, N, R, M, D, L, S, or P, the 2nd X is G, V, K, T, P, R, M, N, E, D, S, C, W, or H, the 3rd X is G, R, E, F, A, K, P, N, L, V, or M, the 4th X is V, D, L, M, S, T, A, N, H, G, F, E, or R, the 5th X is V, K, Y, T, N, G, D, E, P, F, Q, or H, the 6th X is Y, R, H, L, P, N, E, M, A, G, or V, the 7th X is S, K, M, G, H, Q, T, V, C, L, N, or A, the 8th X is C, S, A, T, R, E, N, Q, G, K, Y, or P, the 9th X is E, K, S, V, L, R, G, I, F, T, A, or H, the 10th X is W or G, the 13st X is V, M, D, Y, K, L, I, or R, the 14th X is Y, M, R, S, K, H, G, L, N, or E, the 15th X is Y, W, G, L, N, D, Q, E, M, F, or R, the 16th X is K, the 17th X is M, E, W, V, H, S, N, I, Q, or Y, the 18th X is R, L, V, S, H, Y, F, N, or K, and the 19th X is Q, G, Y, R, N, I, S, H, E, M, L, or D.

4. The synthetic peptide according to claim 3, wherein said amino acid sequence (SEQ ID NO: 1) is any amino acid sequence selected from the group consisting of SEQ ID Nos: 4 to 26.

5. The synthetic peptide according to claim 2, wherein the 1st X from the N-terminus of said amino acid sequence (SEQ ID NO: 2) is A, Q, V, G, R, L, S, K, or E, the 2nd X is G, V, K, T, P, R, N, E, D, S, H, or A, the 3rd X is E, F, A, P, N, V, M, D, H, T, or Y, the 4th X is V, L, M, S, T, H, F, E, R, P, Y, or K, the 5th X is V, T, or I, the 6th X is R, L, M, G, V, or I, the 7th X is S, M, G, T, L, N, A, E, or R, the 8th X is S, T, R, Q, G, L, V, or M, the 9th X is K, S, V, L, R, T, A, Q, N, M, or Y, the 10th X is G, the 11th X is K, the 12th X is L, the 13th X is V, M, L, I, S, Q, G, or E, the 14th X is M, S, H, G, L, or V, and the 15th X is Y, L, Q, E, M, F, R, H, S, K, or V, the 17th X is E, W, V, H, N, Q, G, L, or R, the 18th X is R, S, H, N, Q, W, E, I, or G, and the 19th X is Q, G, R, H, E, L, D, C, W, or V.

6. The synthetic peptide according to claim 5, wherein said amino acid sequence (SEQ ID NO: 1) is any amino acid sequence selected from the group consisting of SEQ ID NOs: 27 to 46.

7. The synthetic peptide according to claim 1, having, in the molecular structure thereof, an amino acid sequence represented by the following (SEQ ID NO: 3), or an amino acid sequence formed by substitution, deletion and/or addition of one or several amino acid residues in the amino acid sequence:
(SEQ ID No. 3)
C A X X L X X L X X X L X X L X G X X X X X
X X X X X X L X X L X X X L X X L X X A C
• • •
in the formula, the 1st X from the N-terminus of sequence (SEQ ID NO: 3) is A, R, Q, V, L, K, F, or H, the 2nd X is E or H, the 3rd X is A, Q, V, Y, R, L, or E, the 4th X is A or S, the 5th X is E, L, H, R, G, or K, the 6th X is A, K, M, R, G, or Y, the 7th X is E or D, the 8th X is A, Q, R, Y, E, K, or G, the 9th X is A or I, the 10th X is E, G, Y, Q, R, K, or M, the 11th X is G or P, the 12th X is G, P, D, or S, the 13th X is G or R, the 14th X is V, G, or P, the 15th X is G or H, the 16th X is L, G, or S, the 17th X is P or R, the 18th X is R or P, the 19th X is L or R, the 20th X is G, the 21st X is K, F, or H, the 22nd X is M, Y, L, R, Q, G, H, F, N, or A, the 23rd X is R, L, N, D, or A, the 24th X is H, F, Y, K, Q, M, or V, the 25th X is Y, L, F, R, V, or I, the 26th X is K or Q, the 27th X is H, Y, L, F, or A, the 28th X is L, N, E, G, or A, the 29th X is F, R, D, Q, M, K, L, or H, and the 30th X is G, N, I, H, L, W, V, or F.

8. The synthetic peptide according to claim 7, wherein whether the 17th and 18th Xs from the N-terminus of said amino acid sequence (SEQ ID NO: 1) are P and the 19th X is L, or all of the 17th to 19th Xs are R.

9. The synthetic peptide according to claim 7, wherein said amino acid sequence (SEQ ID NO: 1) is the amino acid sequence of SEQ ID NO: 47 or 48.

10. The synthetic peptide according to claim 1, comprising any amino acid sequence selected from the group consisting of SEQ ID Nos: 4 to 48, or an amino acid sequence formed by substitution, deletion and/or addition of one or several amino acid residues in the amino acid sequence.

11. The synthetic peptide according to claim 1, wherein WQPPRARIG (SEQ ID NO: 49) is bound through a linker or directly.

12. The synthetic peptide according to claim 1, wherein said synthetic peptide is dimerized.

13. The synthetic peptide according to claim 1, wherein the end of said synthetic peptide is molecularly modified.

14. A composition for inducing differentiation of a pluripotent stem cell into a somatic cell, comprising the synthetic peptide according to claim 1.

15. The composition according to claim 14, further comprising one or more selected from the group consisting of activin, bFGF, BMP4, VEGF, IWP-3, transferrin, and an extracellular matrix.

16. The composition according to claim 14, wherein said somatic cell is an ectodermal, mesodermal, or endodermal cell.

17. The composition according to claim 16, wherein said ectodermal cell is a neuron, said mesodermal cell is a cardiomyocyte, and said endodermal cell is a hepatocyte.

18. The composition according to claim 14 which is a culture medium or a culture substrate.

19-30. (canceled)
